(19) United States
(12) Patent Application Publication
(10) Pub. No.: US 2026/0355293 A1
(43) Pub. Date: Nov. 12, 2026

(54) Intradiscal Treatment of the Cartilage Endplate for Improving Solute Transport and Disc Nutrition

(71) Applicant: The Regents of the University of California, Oakland, CA (US)

(72) Inventors: Aaron Fields, San Francisco, CA (US); Jeffrey Lotz, San Francisco, CA (US); Frank Szoka, San Francisco, CA (US); Aaron Dolor, San Francisco, CA (US); Mohamed Habib, San Francisco, CA (US)

(21) Appl. No.: 19/119,693

(22) PCT Filed: Dec. 18, 2023
(86) PCT No.: PCT/US2023/084643

§ 371 Date: Apr. 9, 2025

Related U.S. Application Data
(60) Provisional application No. 63/433,641, filed on Dec. 19, 2022.

(51) Int. Cl. A61K 38/48; A61K 9/00; A61K 9/06; A61K 45/06; A61K 47/36

(52) U.S. Cl. CPC A61K 38/4886; A61K 9/0019; A61K 9/06; A61K 45/06; A61K 47/36; C12Y 304/24034

(57) ABSTRACT

Compositions and methods are provided for treating intervertebral disc degeneration in a subject in need thereof. In particular, the methods comprise administering a therapeutically effective amount of one or more matrix-modifying enzymes locally to a cartilage endplate to increase permeability of the cartilage endplate and improve intervertebral disc nutrient transport.

### CROSS-REFERENCE TO RELATED APPLICATION

This application claims benefit under 35 U.S.C. § 119(e) of provisional application 63/433,641, filed Dec. 19, 2022, which application is hereby incorporated by reference in its entirety.

### STATEMENT REGARDING FEDERALLY SPONSORED RESEARCH OR DEVELOPMENT

This invention was made with government support under grant numbers P30 AR066262, RO1 AR070198 and R01 AR063705 awarded by the National Institutes of Health. The government has certain rights in the invention.

### BACKGROUND OF THE INVENTION

Back pain is the leading cause of disability and is a major public health problem worldwide (Fernandez-Moure et al., 2018; Haralson lii & Zuckerman, 2009; Matta et al., 2017; Vassilaki & Hurwitz Dc, 2014). In certain subgroups of back pain patients, intervertebral disc degeneration may cause pain (Chou et al., 2011). Current medical interventions for disc degeneration are surgical in nature and are often unsuccessful, which motivates development of nonoperative alternatives. Nonoperative approaches to regenerate the disc and alleviate pain include delivering genes (Woods B I 2011), growth factors (Bae & Masuda, 2011; Derek G Ju 2022), cells (Kregar Velikonja N,2014, Derek G Ju 2022), or other small molecules (Gawri et al., 2013; Wang Z 2013) to promote cell proliferation, stimulate anabolic activities, and reduce catabolism and inflammation. Importantly, these approaches all require a rich nutrient supply to sustain higher cell numbers or metabolic rates (Fernandez-Moure et al., 2018; Horner & Urban, 2001). However, disc degeneration in humans is typically accompanied by a restricted nutrient supply (Fields et al., 2018; Huang et al., 2014; Jill P. G. Urban 2004), which may limit the clinical utility and effectiveness of these therapies (Huang et al., 2014) (Yong-Can Huang 2014, Zhu Q 2016, Binch 2021, Derek G Ju 2022). Yet, there are no existing treatments to improve disc nutrient supply. Developing treatment strategies to improve disc nutrition may therefore aid in slowing or reversing degeneration, and could enhance the regenerative potential of biologic therapies that increase nutrient demands inside the disc.

### SUMMARY OF THE INVENTION

Compositions and methods are provided for treating intervertebral disc degeneration in a subject in need thereof. In particular, the methods comprise administering a therapeutically effective amount of one or more matrix-modifying enzymes locally to a cartilage endplate to increase permeability of the cartilage endplate and improve intervertebral disc nutrient transport.

In one aspect, a method of treating intervertebral disc degeneration in a subject in need thereof is provided, the method comprising administering a therapeutically effective amount of one or more matrix-modifying enzymes locally to a cartilage endplate (CEP).

In certain embodiments, the treatment increases permeability of the CEP and intervertebral disc nutrient transport.

In certain embodiments, one or more of the matrix-modifying enzymes have proteolytic activity against collagen, a proteoglycan, or a combination thereof. In some embodiments the one or more matrix-modifying enzymes comprise a collagenase, wherein the collagenase has proteolytic activity against collagen I, collagen II, collagen III, collagen IV, or any combination thereof. In some embodiments, the collagenase is collagenase P, matrix metalloproteinase 8 (MMP8), or a combination thereof. In some embodiments, the MMP8 is full-length MMP8. In some embodiments, the treatment comprises administering more than one matrix-modifying enzyme.

In certain embodiments, one or more matrix-modifying enzymes are encapsulated in a hydrogel. In some embodiments, the one or more matrix-modifying enzymes encapsulated in the hydrogel comprise a collagenase. In some embodiments, the collagenase is collagenase P, MMP8, or a combination thereof. In some embodiments, the MMP8 is full-length MMP8. In some embodiments, the hydrogel sustains delivery of the one or more matrix-modifying enzymes for at least 2 days. In some embodiments, the hydrogel comprises alginate. In some embodiments, the alginate concentration in the hydrogel ranges from 0.5 to 10 percentage by weight (wt %). In some embodiments, the alginate is at least partially oxidized. In some embodiments, about 4% to about 10% of the alginate is oxidized.

In certain embodiments, the one or more matrix-modifying enzymes are encapsulated in a liposome or conjugated to the surface of a liposome. In some embodiments, the one or more matrix-modifying enzymes, encapsulated in the liposome or conjugated to the surface of the liposome, comprise a collagenase. In some embodiments, the collagenase is collagenase P, MMP8, or a combination thereof. In some embodiments, the MMP8 is full-length MMP8. In some embodiments, the liposome comprises a phospholipid. Exemplary phospholipids include, without limitation, phosphatidylcholine and phosphatidylethanolamine. In some embodiments, the liposome further comprises cholesterol. In some embodiments, the liposome further comprises polyethylene glycol.

In certain embodiments, the one or more matrix-modifying enzymes are administered in a volume sufficiently small such that intradiscal pressure is not substantially increased. In some embodiments, the one or more matrix-modifying enzymes are administered in a volume of no more than 50 μL.

In certain embodiments, the one or more matrix-modifying enzymes are administered into or adjacent to the CEP.

In certain embodiments, the one or more matrix-modifying enzymes are administered with an epidural needle. For example, the epidural needle may have a curved end such that the matrix-modifying enzymes exits the epidural needle laterally at a 45-degree angle.

In certain embodiments, the method further comprises using medical imaging to guide said administering the one or more matrix-modifying enzymes locally to the CEP or for selection of which intervertebral discs may benefit from treatment with the one or more matrix-modifying enzymes. For example, the one or more matrix-modifying enzymes may be administered locally to a target CEP using fluoroscopic guidance, computed tomographic (CT) guidance, combined CT-fluoroscopic guidance, or ultrasound guidance.

In certain embodiments, the one or more matrix-modifying enzymes are administered locally to the CEP without exposing the nucleus pulposus to the one or more matrix-modifying enzymes.

In certain embodiments, the method further comprises administering a growth factor.

In certain embodiments, the method further comprises administering an analgesic agent or an anti-inflammatory agent.

In certain embodiments, multiple cycles of treatment are administered to the subject.

In another aspect, a composition comprising one or more matrix-modifying enzymes for use in a method of treating intervertebral disc degeneration is provided, the method comprising administering a therapeutically effective amount of the one or more matrix-modifying enzymes locally to a cartilage endplate (CEP).

In certain embodiments, the composition further comprises a pharmaceutically acceptable excipient.

In certain embodiments, the one or more of the matrix-modifying enzymes in the composition have proteolytic activity against collagen, a proteoglycan, or a combination thereof. In some embodiments the one or more matrix-modifying enzymes comprise a collagenase, wherein the collagenase has proteolytic activity against collagen I, collagen II, collagen III, collagen IV, or any combination thereof. In some embodiments, the collagenase is collagenase P, MMP8, or a combination thereof. In some embodiments, the MMP8 is full-length MMP8.

In certain embodiments, the one or more matrix-modifying enzymes in the composition are encapsulated in a hydrogel. In some embodiments, the hydrogel comprises alginate. In some embodiments, the alginate concentration in the hydrogel ranges from 0.5 to 10 percentage by weight (wt %). In some embodiments, the alginate is at least partially oxidized. In some embodiments, about 4% to about 10% of the alginate is oxidized.

In certain embodiments, the one or more matrix-modifying enzymes in the composition are encapsulated in a liposome or conjugated to the surface of a liposome. In some embodiments, the liposome comprises a phospholipid. Exemplary phospholipids include, without limitation, phosphatidylcholine and phosphatidylethanolamine. In some embodiments, the liposome further comprises cholesterol. In some embodiments, the liposome further comprises polyethylene glycol.

In certain embodiments, the composition further comprises a growth factor.

In certain embodiments, the composition further comprises an analgesic agent or an anti-inflammatory agent.

In another aspect, a method of increasing intervertebral disc solute transport in a subject in need thereof is provided, the method comprising administering an effective amount of one or more matrix-modifying enzymes locally to a cartilage endplate (CEP).

In certain embodiments, prior to said administering the one or more matrix-modifying enzymes locally to the CEP, the method further comprises detecting if intervertebral disc solute transport is impeded in the CEP to determine if the subject will benefit from the treatment.

### BRIEF DESCRIPTION OF THE DRAWINGS

FIGS. 1A-1B. Intradiscal delivery of collagenolytic enzyme treatment via 17G Tuohy needle to superior CEP of (FIG. 1A) bovine coccygeal and (FIG. 1B) human lumbar intervertebral discs under fluoroscopic guidance.

FIGS. 2A-2B. Experimental setup (FIG. 2A) and the compression testing protocol (FIG. 2B) of the whole intact disc while being immersed in fluorescein tracer solution.

FIGS. 3A-3D. (FIG. 3A) Prior to compression testing, whole discs were isolated from the surrounding soft tissue, and the bony endplate was removed with a burring tool. (FIG. 3B) After compression testing, discs were snap-frozen in liquid nitrogen and two 5 mm-wide mid-sagittal slabs were removed for further biochemical and fluorescein measurement. Circles indicate the locations of CEP punches harvested for biochemical measurements. (FIG. 3C) Mid-sagittal slab showing NP tissue sample positions (three 2 mm punches per inferior-superior positions) relative to treated and un-treated CEPs. (FIG. 3D) Two 4 mm-diameter punches were harvested from the central region of each CEP in the mid-sagittal slab (FIG. 3B).

FIGS. 4A-4B. (FIG. 4A) The average fluorescein concentration (mean±SD; n=3 discs/group) was significantly higher in the bovine NP proximal to the collagenase-treated superior CEP, position A. (a p<0.0001 vs. control buffer; b p<0.0001 vs. sham; p>0.10 all other comparisons). (FIG. 4B) The average fluorescein concentration (mean±SD; n=5 discs/group) was significantly higher in the human NP proximal to the collagenase-treated superior CEP, position A. (a p<0.0001).

FIGS. 5A-5B. (FIG. 5A) In the bovine discs that underwent collagenase treatment, sGAG content in the treated (superior) CEP was significantly lower than the sGAG content in the untreated (inferior) CEPs (p=0.03, inferior versus superior paired t-test; mean±SD; n=3 discs/group) (FIG. 5B) In the human discs that underwent collagenase treatment, sGAG content in the treated (superior) CEPs was significantly lower than the sGAG content in the untreated (inferior) CEPs (p<0.01, inferior versus superior paired t-test; mean±SD; n=5 discs/group).

FIGS. 6A-6B. (FIG. 6A) In the bovine discs that underwent collagenase treatment, collagen content in the treated (superior) CEPs was similar to that of the untreated (inferior) CEPs (p=0.21, paired t-test; mean±SD; n=3 discs/group) (FIG. 6B) In the human discs that underwent collagenase treatment, collagen content in the treated (superior) CEPs was similar to that in the untreated (inferior) CEPs (p=0.07, paired t-test; mean±SD; n=5 discs/group).

FIGS. 7A-7B. Whole-disc compressive creep strain (top) and modulus (bottom was similar in (FIG. 7A) bovine and (FIG. 7B) human discs with collagenase-treated CEPs vs. control CEPs. p-values are from one-way ANOVA.

FIG. 8 Scatterplot showing the relationship between sGAG content in the human CEP and fluorescein concentration in the adjacent NP tissue. For the collagenase-treated CEPs, each marker represents the average sGAG content in one of the four CEP biopsies harvested from the central region of the superior (treated) CEP and the average fluorescein concentration in the site-matched NP tissue at position A. Error bars show SEM for 5 discs. For the control CEPs, each marker represents the average sGAG content in one of the four CEP biopsies harvested from the central region of the superior and inferior non-treated CEP and the average fluorescein concentration in the site-matched NP tissue.

FIG. 9. 40 kDa dextran cumulative release (%) of alginate gels with 10%, 5% and 0% oxidation over 24 h. Different alginate gels mixed with the fluorescent dextran (50 μL total volume) were placed in opaque Eppendorf tubes containing 2 mL ddH2O, and aliquots were assayed for fluorescence at 0.2, 1, 2, 4, 20 and 24 h using a microplate reader (494 nm/524 nm ex/em).

FIGS. 10A-10C. CEP of bovine disc was superficially treated with collagenase or control buffer (n=3 discs/group). Following treatment, discs were compressed between porous platens while submerged in fluorescein tracer solution (376 Da; 0.1 mg/mL). At the end of the compression testing, discs were flash-frozen in liquid nitrogen, slabbed coronally, and assayed for fluorescein concentration. (FIG. 10A) Fluorescein concentration was 3.3-fold higher in the NP proximal to the collagenase-treated CEP. (FIG. 10B) Collagenase treated CEPs showed greater sGAG release during the 18 hr treatment period than buffer-treated CEPs (p=0.01), which coincided with higher fluorescein levels at position A. (FIG. 10C) Whole-disc creep behavior was similar for discs with collagenase-treated and buffer treated CEPs.

FIG. 11: Collagen content in the CEP after treatment. n=3-4 samples/group; mean±SEM.

FIG. 12: Concentration ratios, C=C_{downstream}/Cᵤₚₛₜᵣₑₐₘ, from pilot tests (0.6-1.0 MPa@0.5 Hz for 80 min) of CEPs treated with full-length MMP-8 or buffer.

FIG. 13: Effects of MMP-8 proteolysis on cytokine production by CEP cells after 72 hr (fold differences relative to “basal” control). Mean±SEM for 5 patients. Releasates from proteolysis by full-length MMP-8 (0.2 U/mL [200 ng/mL]; 2.0 U/mL [2000 ng/mL]). \hat{ }p<0.05 vs. basal.

FIGS. 14A-14B: (FIG. 14A) Cell viability in NP and CEP tissues from bovine discs with treated CEPs (mean±SD for 5 projections per disc, 1 disc per group). “Day 0” shows mean±SD for 5 projections per disc for 2 discs. (FIG. 14B) Fluorescein concentration (mean±SEM for 12 samples, 6 per position for each disc×2 discs/group) was significantly higher in NP near MMP-8-treated CEP, position A (p<0.0001). NP tissue positions A-D correspond to FIG. 12C.

### DETAILED DESCRIPTION OF THE INVENTION

Compositions and methods are provided for treating intervertebral disc degeneration in a subject in need thereof. In particular, the methods comprise administering a therapeutically effective amount of one or more matrix-modifying enzymes locally to a cartilage endplate to increase permeability of the cartilage endplate and improve intervertebral disc nutrient transport.

Before the present methods, devices, and compositions are described, it is to be understood that this invention is not limited to particular methods, devices, or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein the singular forms “a”, “an”, and “the” include plural referents unless the context clearly dictates otherwise. Thus, for example, reference to “a disc” includes a plurality of such discs and reference to “the drug” includes reference to one or more drugs and equivalents thereof (e.g., therapeutics, medicines, medicaments), known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “about,” particularly in reference to a given quantity, is meant to encompass deviations of plus or minus five percent.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of an active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease, or which improves resistance to a disorder.

The terms “pharmacologically effective amount” or “therapeutically effective amount” of a matrix-modifying enzyme refers to an amount of the matrix-modifying enzyme sufficient to provide the desired response, such as improved nutrient transport through the cartilage endplate. In addition, a therapeutically effective amount of a matrix-modifying enzyme may prevent, slow development of, or reduce intervertebral disc degeneration, ameliorate pain caused by disc degeneration, improve spinal movement, improve spinal flexibility, and/or increase disc height and/or the separation distance between vertebrae. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Treatment” or “treating” intervertebral disc degeneration includes: (1) preventing intervertebral disc degeneration, i.e., causing intervertebral disc degeneration not to develop or to occur with less severity in a subject that may be predisposed to develop intervertebral disc degeneration but does not yet experience or display intervertebral disc degeneration, (2) inhibiting intervertebral disc degeneration, i.e., arresting the development, slowing, or reversing intervertebral disc degeneration, (3) relieving symptoms of intervertebral disc degeneration, i.e., decreasing the severity of intervertebral disc degeneration, improving spine flexibility, or reducing back pain experienced by the subject.

“Substantially” or “essentially” means nearly totally or completely, for instance, 95% or greater of some given quantity.

“Substantially purified” generally refers to isolation of a substance (compound, polynucleotide, protein, polypeptide, peptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

As used herein, the terms “increase”, “increasing”, “enhance”, and “enhancing” (and grammatical variations thereof) describes, unless the context indicates otherwise, a detectable elevation compared to a reference value. An increase can comprise an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include post-expression modifications of the polypeptide, for example, phosphorylation, glycosylation, acetylation, hydroxylation, oxidation, and the like.

As used herein, “reference sequence” is a defined sequence used as a basis for sequence comparison. A reference sequence may be a subset or the entirety of a specified sequence; for example, as a segment of a full-length protein or protein fragment. A reference sequence can comprise, for example, a sequence identifiable in a database such as GenBank and UniProt and others identifiable to those skilled in the art.

Algorithms and programs for comparing primary biological sequence information between any two sequences are identifiable by a skilled person. Non-limiting examples of such mathematical algorithms are the algorithm of Myers and Miller (Myers and Miller 1988), the local homology algorithm of Smith et al. (Smith and Waterman 1981); the homology alignment algorithm of Needleman and Wunsch (Needleman and Wunsch 1970)); the search-for-similarity-method of Pearson and Lipman (Pearson and Lipman 1988).); the algorithm of Karlin and Altschul (Karlin and Altschul 1990), modified as in Karlin and Altschul (Karlin and Altschul 1993)). Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA (Pearson and Lipman 1988).); and TFASTA in the Wisconsin Genetics Software Package, Version 8 (available from Genetics Computer Group (GCG), 575 Science Drive, Madison, Wis., USA). Alignments using these programs can be performed using the default parameters and allowing a user to identify database sequences that resemble the reference sequence (query sequence) above a certain threshold of confidence.

Algorithms and programs for comparing primary biological sequence information between any two sequences typically provide an output comprising percent identity between the sequence retrieved and the reference sequence.

A person skilled in the art would understand that identity between sequences is typically measured by a process that comprises the steps of aligning the two polypeptide or polynucleotide sequences to form aligned sequences, then detecting the number of matched characters, i.e. characters similar or identical between the two aligned sequences, and calculating the total number of matched characters divided by the total number of aligned characters in each polypeptide or polynucleotide sequence, including gaps. The similarity result is expressed as a percentage of identity.

As used herein, “percentage of sequence identity” means the value determined by comparing two optimally aligned sequences over a comparison window, wherein the portion of the polynucleotide sequence in the comparison window may comprise additions or deletions (gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The phrase “pharmaceutically acceptable” refers to molecular entities and compositions that are physiologically tolerable and do not typically produce an allergic or similar untoward reaction, such as gastric upset, dizziness and the like, when administered to a human (or non-human animal in the case of veterinary use). Preferably, as used herein, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Biocompatible,” as used herein, refers to a property of a material that allows for prolonged contact with a cell or tissue without causing toxicity or significant damage.

The term “hydrogel” refers to a substance formed when an organic polymer (natural or synthetic) is cross-linked via covalent, ionic, or hydrogen bonds to create a three-dimensional open-lattice structure which entraps water molecules to form a gel. In general, these polymers are at least partially soluble in aqueous solutions, such as water, buffered salt solutions, or aqueous alcohol solutions that have charged side groups, or a monovalent ionic salt thereof. A biocompatible hydrogel refers to a polymer that forms a gel which is not toxic to living cells.

By “subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

### Increasing Intervertebral Disc Solute Transport

Compositions and methods are provided for increasing intervertebral disc solute transport. Poor nutrient transport through the cartilage endplate contributes to intervertebral disc degeneration and limits the effectiveness of therapies to promote disc regeneration. Nutrients entering the disc and exiting metabolites must pass through the cartilage endplates. Solute passage may be slowed or blocked by changes to the composition of the CEP matrix caused, for example, by dehydration, mineralization, and/or fibrosis. Intradiscal delivery of a matrix-modifying enzyme to the cartilage endplate improves solute transport and disc nutrition, which may aid in slowing or reversing disc degeneration, and enhance the regenerative potential of biologic therapies, particularly those that increase nutrient demands inside the disc.

In some embodiments, a method of increasing intervertebral disc solute transport in a subject in need thereof is provided, the method comprising administering an effective amount of one or more matrix-modifying enzymes locally to a cartilage endplate. In certain embodiments, the one or more matrix-modifying enzymes have proteolytic activity against collagen, a proteoglycan, and/or a combination thereof. In some embodiments the one or more matrix-modifying enzymes comprise a collagenase, wherein the collagenase has proteolytic activity against collagen I, collagen 1l, collagen Ill, collagen IV, or any combination thereof. Exemplary collagenases include, without limitation, microbial collagenases such as bacterial collagenases from Vibrio (e.g., Vibrio alginolyticus, Vibrio pomeroyi, Vibrio mimicus, and Vibrio Grimontia) and Clostridium (e.g., Clostridium histolyticum and Clostridium perfringens) bacteria; and matrix metalloproteinases (MMPs) with collagenase activity such as, but not limited to, MMP1, MMP8, MMP13, and MMP18. In some embodiments, the collagenase is collagenase P, MMP8, or a combination thereof. In some embodiments, the collagenase is collagenase P from Clostridium histolyticum. In some embodiments, the MMP8 is full-length MMP8. Enzymatically reducing the amounts of collagen in impermeable cartilage endplates improves disc solute transport and nutrition. Collagenases can be derived from an organism, produced recombinantly, or synthesized chemically by methods well known in the art. A number of collagenases are commercially available, for example, from Nordmark Pharma GmbH (Uetersen, Germany), VitaCyte LLC (Indianapolis, IN), Universal Biologicals (Cambridge, United Kingdom), Thermo Fisher Scientific (Waltham, MA), and Worthington Biochemical (Lakewood, New Jersey).

In some embodiments, one or more matrix-modifying enzymes are encapsulated in a hydrogel. Any suitable hydrogel polymers can be used to form a hydrogel. Exemplary hydrogel polymers include, without limitation, natural polymers such as polysaccharides, including hyaluronic acid, chitosan, heparin, alginate, cellulose, dextran, and agarose, and proteins, including fibrin, fibrinogen, collagen, elastin, gelatin, silk, laminin, fibronectin, albumin, thrombin, and keratin; modified natural polymers, including hydroxymethylcellulose, hydroxyethylcellulose, gelatin methacrylate, polyanionic N-carboxymethyl chitosan, and polycationic N-trimethyl chitosan; and synthetic polymers, including polyvinyl alcohol, N-vinylpyrrolidone, polyethylene glycol, poly(ethylene glycol) diacrylate, polyacrylamide, poly(N-isopropylacrylamide), sodium polyacrylate, acrylate polymers and copolymers such as hydroxyethyl methacrylate, ethyl methacrylate, propylene glycol methacrylate, ethylene glycol di-methyl acrylate, methyl methacrylate, glycidyl methacrylate, and glycol methacrylate, poly(N-isopropylacrylamide-co-acrylic acid), polyesters, polyurethanes, nylon, synthetic polyamino acids, prolamines; and combinations thereof, and other such molecules, including recombinant versions of such polymers.

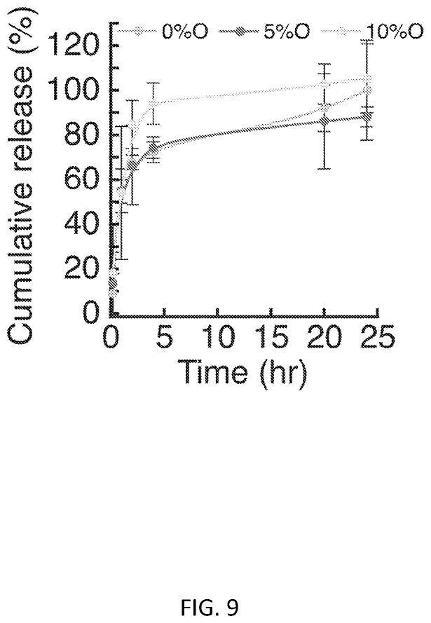

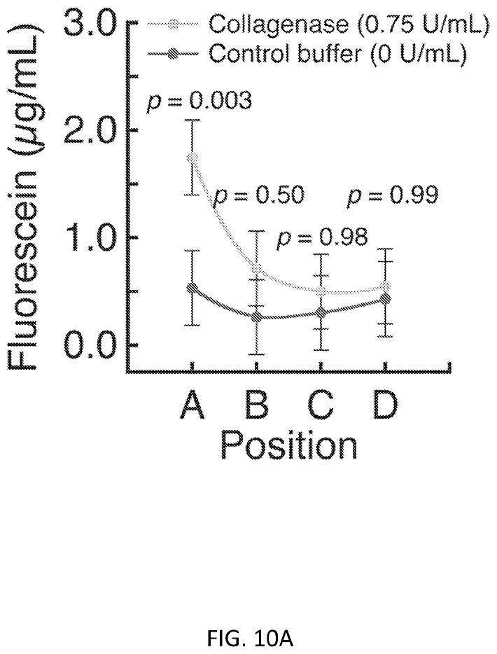

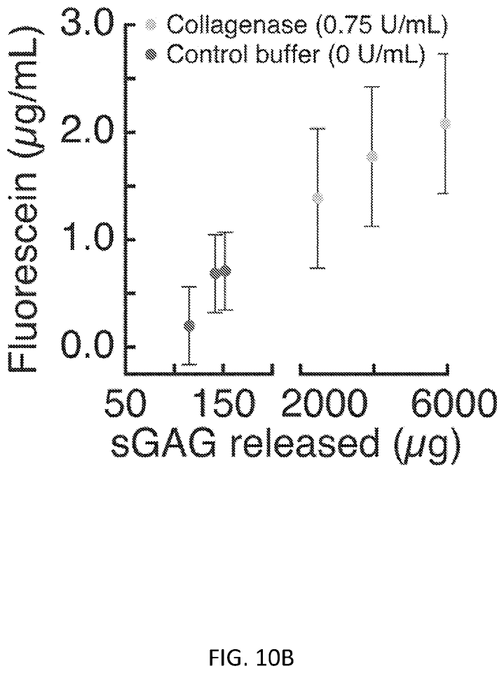

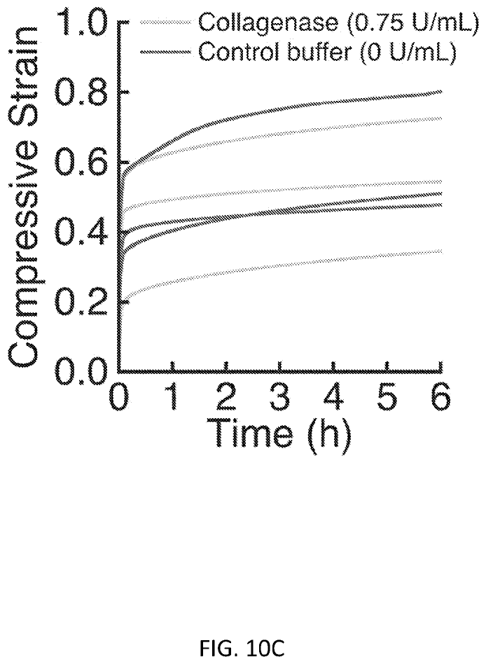

In an exemplary embodiment, one or more matrix-modifying enzymes are encapsulated in an alginate hydrogel (see, e.g., Example 1 for a description of a biodegradable alginate hydrogel encapsulating collagenase P from Clostridium histolyticum). In some embodiments, the alginate concentration in the hydrogel ranges from about 0.5 to about 10 percentage by weight (wt %), including any wt % within this range, such as 0.5, 1, 2, 2.5, 3, 3.5, 4, 4.5, 5, 5.5, 6, 6.5, 7, 7.5, 8, 8.5, 9, 9.5, or 10 wt %. In some embodiments, the alginate is partially oxidized. For example, about 2% to about 10% of the alginate may be oxidized, including any percent in this range, such as 2%, 2.5%, 3%, 3.5%, 4%, 4.5%, 5%, 5.5%, 6%, 6.5%, 7%, 7.5%, 8%, 8.5%, 9%, 9.5%, or 10%. In one embodiment, the alginate in the hydrogel is about 4% to about 10% oxidized. In some embodiments, the alginate is at a concentration of about 1 wt % in the hydrogel. Such alginate hydrogels are capable of sustaining delivery of a matrix-modifying enzyme for at least 2 days after administration to a subject (see, e.g., Example 1 and FIG. 9).

Methods of preparing hydrogels are well known in the art. See, e.g., Barbucci Hydrogels Biological Properties and Applications, Springer, 2009; Hydrogels in Cell-Based Therapies, edited by Connon and Hamley, Royal Society of Chemistry, 2014; Tunable Hydrogels, edited by Lavrentieva, Pepelanov, and Seliktar, Springer Nature Switzerland AG, 2020; herein incorporated by reference. In certain instances, a hydrogel is crosslinked by chemical crosslinking. Exemplary chemical crosslinking agents include, without limitation, N,N,N′,N′-tetramethylethylenediamine (TEMED), ammonium persulfate, glutaraldehyde, formaldehyde, epoxy compounds, dialdehyde, N,N′-methylenebis(acrylamide) (MBA), ethylene glycol diacrylate (EGDA), ethylene glycol dimethylacrylate (EGDMA), PEG diacrylate (PEGDA), glyoxal, epichlorohydrin, and sodium borate/boric acid. The crosslink density may vary depending on the type and the concentration of the chemical crosslinking agent employed. Alternatively, the hydrogel may be photo-crosslinked by exposure to light. UV light-sensitive photoinitiators (i.e., initiate polymerization with exposure to UV light in the range of 200-400 nm) or visible light-sensitive photoinitiators (i.e., initiate polymerization with exposure to visible light in the range of 400-800 nm) may be used. A photoinitiator may be used to induce photopolymerization. Photopolymerization reactions may involve a free-radical-initiated chain polymerization or bio-orthogonal click reactions. Exemplary photoinitiators include, without limitation, (1-[4-(2-hydroxyethoxy)-phenyl]-2-hydroxy-2-methyl-1-propane-1-one), lithium phenyl-2,4,6-trimethylbenzoylphosphinate (LAP), and Eosin-Y. The crosslink density may vary depending on the intensity of the electromagnetic radiation applied to promote photopolymerization of the hydrogel as well as the duration of irradiation. In some embodiments, the crosslink density of the crosslinked hydrogel ranges from 1×10⁻¹⁵ moles/cm³ to 1×10⁻³ moles/cm³. In some embodiments, the hydrogel is ionically cross-linked with divalent cations (e.g., Ca²⁺, Mg²⁺, Zn²⁺, or Ba²⁺) or trivalent cations (e.g., Fe³⁺ or Al³⁺).

In other embodiments, one or more matrix-modifying enzymes are encapsulated in a liposome or conjugated to the surface of a liposome. Liposomal preparations for use in delivering a matrix-modifying enzyme may include cationic (positively charged), anionic (negatively charged), and neutral lipids. Various cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Felgner et al., Proc. Natl. Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes. Anionic and neutral liposomes are also readily available, such as, from Avanti Polar Lipids (Birmingham, AL), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with DOTMA and DOTAP in appropriate ratios. Methods for making liposomes using these materials are well known in the art. The type of liposome selected is preferably of a size and composition suitable for limiting mobility of matrix-modifying enzymes into the nucleus pulposus.

Any suitable bioconjugation method may be used for conjugating a matrix-modifying enzyme to the surface of a liposome. Crosslinking agents that can be used include, but are not limited to, dimethyl suberimidate, N-hydroxysuccinimide, formaldehyde, and glutaraldehyde. In addition, carboxyl-reactive chemical groups such as diazomethane, diazoacetyl, and carbodiimide can be included for crosslinking carboxylic acids to primary amines. In particular, the carbodiimide compounds, 1-ethyl-3-(-3-dimethylaminopropyl) carbodiimide hydrochloride (EDC) and N′,N′-dicyclohexyl carbodiimide (DCC) can be used for conjugation with carboxylic acids. In order to improve the efficiency of crosslinking reactions, N-hydroxysuccinimide (NHS) or a water-soluble analog (e.g., Sulfo-NHS) may be used in combination with a carbodiimide compound. The carbodiimide compound (e.g., EDC or DCC) couples NHS to carboxyl groups to form an NHS ester intermediate, which readily reacts with primary amines at physiological pH. For a description of various crosslinking agents and techniques, see, e.g., Wong and Jameson Chemistry of Protein and Nucleic Acid Cross-Linking and Conjugation (CRC Press, 2^{nd} edition, 2011), Hermanson Bioconjugate Techniques (Academic Press, 3^{rd} edition, 2013), herein incorporated by reference in their entireties.

In certain embodiments, a matrix-modifying enzyme is crosslinked to the surface of a liposome using click chemistry. Crosslinking with click chemistry can be performed with suitable crosslinking agents comprising reactive azide or alkyne functional groups. See, e.g., Kolb et al., 2004, Angew Chem Int Ed 40:3004-31; Evans, 2007, Aust J Chem 60:384-95; Millward et al. (2013) Integr Biol (Camb) 5(1):87-95, Lallana et al. (2012) Pharm Res 29(1):1-34, Gregoritza et al. (2015) Eur J Pharm Biopharm. 97(Pt B):438-453, Musumeci et al. (2015) Curr Med Chem. 22(17):2022-2050, McKay et al. (2014) Chem Biol 21(9):1075-1101, Ulrich et al. (2014) Chemistry 20(1):34-41, Pasini (2013) Molecules 18(8):9512-9530, and Wangler et al. (2010) Curr Med Chem. 17(11):1092-1116; herein incorporated by reference in their entireties.

In particular, crosslinking can be performed using strain-promoted azide-alkyne cycloaddition (SPAAC) click chemistry, a Cu-free variation of click chemistry that is generally biocompatible with cells. SPAAC utilizes a substituted cyclooctyne having an internal alkyne in a strained ring system. Ring strain together with electron-withdrawing substituents in the cyclooctyne promote a [3+2] dipolar cycloaddition with an azide functional group. SPAAC can be used for bioconjugation and crosslinking by attaching azide and cyclooctyne moieties to molecules. For a description of SPAAC, see, e.g., Baskin et al. (2007) Proc Natl Acad Sci USA 104(43):16793-16797, Agard et al. (2006) ACS Chem. Biol. 1: 644-648, Codelli et al. (2008) J. Am. Chem. Soc. 130:11486-11493, Gordon et al. (2012) J. Am. Chem. Soc. 134:9199-9208, Jiang et al. (2015) Soft Matter 11(30):6029-6036, Jang et al. (2012) Bioconjug Chem. 23(11):2256-2261, Ornelas et al. (2010) J Am Chem Soc. 132(11):3923-3931; herein incorporated by reference in their entireties.

A medical practitioner may locate the target CEP for treatment with a matrix modifying enzyme, for example, by medical imaging. Medical imaging may also be used for selection of which intervertebral discs may benefit from treatment with a matrix-modifying enzyme. Exemplary medical imaging techniques include, without limitation, magnetic resonance imaging (MRI), positron emission tomography (PET), single photon emission computed tomography (SPECT), computed tomography (CT), ultrasound imaging (UI), optical imaging (O), photoacoustic imaging (PI), fluoroscopy, and fluorescence imaging. In some embodiments, the one or more matrix-modifying enzymes are administered locally to a target CEP using fluoroscopic guidance, computed tomographic (CT) guidance, combined CT-fluoroscopic guidance, or ultrasound guidance.

### Pharmaceutical Compositions

One or more matrix-modifying enzymes (e.g., collagenase, full-length MMP8) can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, buffers, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof. Those skilled in the art will readily recognize a variety of buffers that could be used in the compositions. Buffers include, but are not limited to, pharmaceutically acceptable weak acids, weak bases, or mixtures thereof. For example, the buffer components can be water soluble materials such as phosphoric acid, tartaric acids, lactic acid, succinic acid, citric acid, acetic acid, ascorbic acid, aspartic acid, glutamic acid, and salts thereof. Exemplary buffering agents include, for example, a Tris buffer, N-(2-hydroxyethyl)piperazine-N′-(2-ethanesulfonic acid) (HEPES), 2-(N-morpholino)ethanesulfonic acid (MES), 2-(N-morpholino)ethanesulfonic acid sodium salt (MES), 3-(N-Morpholino)propanesulfonic acid (MOPS), and N-tris[hydroxymethyl]methyl-3-aminopropanesulfonic acid (TAPS).

A composition can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the one or more matrix-modifying enzymes, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

In certain embodiments, one or more additional factors, such as nutrients, cytokines, growth factors, or immunosuppressive agents may be added to a composition.

Exemplary growth factors include, without limitation, insulin-like growth factor (IGF), transforming growth factor beta (TGF-β), growth/differentiation factor 5 (GDF-5), osteogenic protein-1, bone morphogenetic proteins (BMPs), epiregulin, fibroblast growth factor (FGF), epidermal growth factor (EGF), endothelial cell growth factor (ECGF), nerve growth factor (NGF), leukemia inhibitory factor (LIF), hepatocyte growth factor (HGF), vascular endothelial growth factor (“VEGF”), and cholecystokinin octapeptide.

Exemplary immunosuppressive agents are well known and may be steroidal (e.g., prednisone) or non-steroidal (e.g., sirolimus (Rapamune, Wyeth-Ayerst Canada), tacrolimus (Prograf, Fujisawa Canada), and anti-IL2R daclizumab (Zenapax, Roche Canada). Other immunosuppressant agents include 15-deoxyspergualin, cyclosporin, methotrexate, rapamycin, Rapamune (sirolimus/rapamycin), FK506, or Lisofylline (LSF).

The amount of a matrix-modifying enzyme in the composition (e.g., when contained in a drug delivery system) will vary depending on a number of factors but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for localized delivery to a CEP.

The pharmaceutical preparations herein can also be housed in a syringe, an implantation device, or the like, depending upon the intended mode of delivery and use. Preferably, the compositions comprising the matrix-modifying enzyme are in unit dosage form, meaning an amount of a conjugate or composition of the invention appropriate for a single dose, in a premeasured or pre-packaged form.

The compositions herein may optionally include one or more additional agents for treating intervertebral disc degeneration such as analgesic or anti-inflammatory agents or other medications. For example, compounded preparations may include at least one matrix-modifying enzyme and one or more other drugs for treating intervertebral disc degeneration such as analgesic and anti-inflammatory agents including, without limitation, acetaminophen, nonsteroidal anti-inflammatory drugs (e.g., aspirin, ibuprofen and naproxen), COX-2 inhibitors (e.g., rofecoxib, celecoxib, and etoricoxib), opioids (e.g., morphine, codeine, oxycodone, hydrocodone, dihydromorphine, and pethidine), steroids (e.g., hydrocortisone, prednisone, triamcinolone, methyl-prednisolone, and dexamethasone), gabapentin, memantine, pregabalin, cannabinoids, tramadol, lamotrigine, carbamazepine, duloxetine, milnacipran, tricyclic antidepressants (e.g., amitriptyline, nortriptyline, and desipramine), and serotonin-norepinephrine reuptake inhibitors (e.g., duloxetine, venlafaxine, and milnacipran).

### Administration of Matrix-Modifying Enzymes

At least one therapeutically effective cycle of treatment with a composition comprising one or more matrix-modifying enzymes (e.g., collagenase, full-length MMP8) will be administered to a subject for treatment of intervertebral disc degeneration. By “therapeutically effective dose or amount” of a matrix-modifying enzyme is intended an amount that, when administered brings about a positive therapeutic response, such as improving nutrient transport through the cartilage endplate into discs. In addition, a therapeutically effective amount of a matrix-modifying enzyme may prevent, slow development of, or reduce intervertebral disc degeneration, ameliorate pain caused by disc degeneration, improve spinal movement, improve spinal flexibility, and/or increase disc height and/or the separation distance between vertebrae. The exact amount required will vary from subject to subject, depending on the species, age, and general condition of the subject, the severity of the condition being treated, the particular type of matrix-modifying enzyme employed, the mode of administration, and the like. An appropriate “effective” amount in any individual case may be determined by one of ordinary skill in the art using routine experimentation, based upon the information provided herein.

The treatment may be administered to one or both CEPs of one or more discs of a vertebrate subject. The discs treated may be any disc of the spinal column. In some embodiments, the subject is a human. In human subjects, there are 25 discs, including 7 cervical discs, 12 thoracic discs, five lumbar discs, and one sacral disc, any of which may be treated. In other embodiments, the subject is a non-human vertebrate animal species. Vertebrate animals may be treated, for example, for veterinary or research purposes. Non-human vertebrate subjects include, but are not limited to, non-human primates such as chimpanzees and other apes and monkey species; farm animals such as cattle, sheep, pigs, goats, and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

In some embodiments, the subject has been diagnosed as having disc degeneration. For example, a subject may be diagnosed using radiographs, computed tomography, magnetic resonance, or discography. In some embodiments, the subject has spinal pain, for example, lower back pain or neck pain.

In other embodiments, the pharmaceutical composition comprising one or more matrix-modifying enzymes is administered prophylactically, e.g., to prevent intervertebral disc degeneration. Such prophylactic uses will be of particular value for subjects at high risk of developing intervertebral disc degeneration because of inadequate nutrient transport through the cartilage endplate, a genetic predisposition to developing intervertebral disc degeneration (e.g., due to mutations in COL1A1, COL9A1, COL9A2, and COL9A3, COL11A1, COL11A2, ACAN, TIMP1, COX2 or THSD2 genes), age, spine deformities, spine injuries, a physically demanding occupation (e.g., requiring carrying heavy loads), obesity, or smoking. In some cases, a subject is at risk of developing disc degeneration because of the age of the subject. For example, in humans, subjects over 45 years of age may be at risk of developing disc degeneration. In some embodiments, the subject is over 45 years of age, over 50 years of age, over 55 years of age, or over 60 years of age.

In certain embodiments, multiple therapeutically effective doses of compositions comprising one or more matrix-modifying enzymes, and/or one or more other therapeutic agents, such as one or more agents for treating intervertebral disc degeneration or other medications are administered. For example, compounded preparations may include at least one matrix-modifying enzyme and one or more drugs for treating intervertebral disc degeneration such as analgesic and anti-inflammatory agents including, without limitation, acetaminophen, nonsteroidal anti-inflammatory drugs (e.g., aspirin, ibuprofen and naproxen), COX-2 inhibitors (e.g., rofecoxib, celecoxib, and etoricoxib), opioids (e.g., morphine, codeine, oxycodone, hydrocodone, dihydromorphine, and pethidine), steroids (e.g., hydrocortisone, prednisone, triamcinolone, methyl-prednisolone, and dexamethasone), gabapentin, memantine, pregabalin, cannabinoids, tramadol, lamotrigine, carbamazepine, duloxetine, milnacipran, tricyclic antidepressants (e.g., amitriptyline, nortriptyline, and desipramine), and serotonin-norepinephrine reuptake inhibitors (e.g., duloxetine, venlafaxine, and milnacipran), or other medications will be administered. The compositions comprising the one or more matrix-modifying enzymes are typically, although not necessarily, administered locally into or adjacent to a CEP in need of treatment.

The pharmaceutical preparation can be in the form of a liquid solution or suspension immediately prior to administration, but may also take another form such as a syrup, cream, ointment, tablet, capsule, powder, gel, liposome, matrix, or the like. The pharmaceutical compositions comprising the one or more matrix-modifying enzymes and/or other agents may be administered using the same or different routes of administration in accordance with any medically acceptable method known in the art.

Compositions comprising one or more matrix-modifying enzymes are suitable for local delivery to a target CEP. A medical practitioner may locate the CEP to be injected, for example, by medical imaging. Exemplary medical imaging techniques include, without limitation, magnetic resonance imaging (MRI), positron emission tomography (PET), single photon emission computed tomography (SPECT), computed tomography (CT), ultrasound imaging (UI), optical imaging (OI), photoacoustic imaging (PI), fluoroscopy, and fluorescence imaging. In some embodiments, the one or more matrix-modifying enzymes are administered locally to a target CEP using fluoroscopic guidance, computed tomographic (CT) guidance, combined CT-fluoroscopic guidance, or ultrasound guidance. In some embodiments, a contrast agent is included in the composition comprising the matrix-modifying enzyme to allow confirmation of localization of the composition to the CEP by medical imaging after administration. In some embodiments, the contrast agent is a microbubble (e.g., for use in ultrasound) or a radiopaque contrast agent (e.g., for use in radiography). The contrast agent may be contained in the same composition as the matrix-modifying enzyme or in a different composition and used prior to or after administration of the matrix-modifying enzyme. In certain embodiments, the composition is injected into the CEP or adjacent to the CEP.

In another embodiment, the pharmaceutical compositions comprising one or more matrix-modifying enzymes, and/or other drugs for treating intervertebral disc degeneration, and/or other agents are in a sustained-release formulation (e.g., slow-release hydrogel carrier), or a formulation that is administered using a sustained-release device. Such devices are well known in the art, and include, for example, miniature implantable pumps that can provide for drug delivery over time in a continuous, steady-state fashion at a variety of doses to achieve a sustained-release effect with a non-sustained-release pharmaceutical composition.

Those of ordinary skill in the art will appreciate which conditions a matrix-modifying enzyme or a derivative thereof can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

In certain embodiments, multiple therapeutically effective doses of a composition comprising one or more matrix-modifying enzymes will be administered according to a daily dosing regimen or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, every other week, and so forth. For example, in some embodiments, a composition comprising the matrix-modifying enzyme will be administered once-weekly, twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., once-weekly, twice-weekly or thrice-weekly administration of a therapeutically effective dose) for one or more weekly cycles until the desired therapeutic response is achieved. The agents can be administered by any acceptable route of administration as noted herein below. The amount administered will depend on the potency of the one or more matrix-modifying enzymes and/or other agents administered, the magnitude of the effect desired, and the route of administration.

The matrix-modifying enzyme (again, preferably provided as part of a pharmaceutical preparation) can be administered alone or in combination with one or more other therapeutic agents, such as other agents for treating intervertebral disc degeneration, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

The one or more matrix-modifying enzymes can be administered prior to, concurrent with, or subsequent to other agents. If provided at the same time as other agents, the one or more matrix-modifying enzymes can be provided in the same or in a different composition. Thus, the one or more matrix-modifying enzymes and one or more other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising the one or more matrix-modifying enzymes and a dose of a pharmaceutical composition comprising at least one other agent, such as another drug for treating intervertebral disc degeneration, which in combination comprise a therapeutically effective dose, according to a particular dosing regimen. Similarly, the one or more matrix-modifying enzymes and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

Toxicity can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

### Diagnostic Methods

Diagnostic methods are also provided for determining whether a subject is in need of treatment to increase intervertebral disc solute transport. The diagnostic methods generally comprise detecting aberrant thickness, abundance, or composition of the cartilage layer in vertebral endplates adjoining degenerating discs as a marker of impeded transport. In one embodiment, the cartilage status (e.g. thickness, abundance, and/or composition) of the endplates adjoining a degenerating disc is assessed in a patient. If one or both of the endplates appears to have aberrant cartilage status, then impeded permeability of the endplate or endplates is implicated in the degenerative disc condition, and indicates a patient may benefit from a permeability-enhancing treatment, as described herein.

In addition, such assessments of endplate cartilage status can be used to monitor the efficacy of a permeability-enhancing treatment. In some embodiments, the status of cartilage in endplates is assessed in a subject undergoing a treatment. A decrease in aberrant cartilage indicators in the treated subject, relative to an appropriate control indicates that the permeability-enhancing treatment is effective. An appropriate control may be used, such as cartilage thickness, abundance, and/or composition in untreated subjects or may be that observed in the same subject at an earlier time point (e.g. prior to or earlier in treatment). This method is useful for monitoring the efficacy of the treatment in an individual patient so that treatment course can be altered if ineffective. Furthermore, this method is useful for assessing the efficacy of a putative treatment, for example in test subjects or test animals.

Assessment of cartilage status may be performed by any imaging modality capable of visualizing and quantifying aberrant cartilage indicators in endplates, such as the thickness or abundance of cartilage or an excess of collagen. For example, in one embodiment, MR imaging of the endplate is utilized to assess cartilage status. The endplate cartilage isn't visible using conventional MRI because it has a short T2 relaxation time, and so the cartilage signal isn't captured by conventional MR sequences with long echo times. To overcome this challenge, an ultrashort echo time (UTE) sequence for MR imaging of the cartilage component of the endplate may be employed. For example, in related research, it was observed that relaxation times derived from UTE images correlate with the water content and collagen content of the cartilage, as described in Fields et al., 2014, “Cartilaginous endplates: quantitative MR imaging with very short echo times—orientation dependence and correlation with biochemical composition” Radiology 274(2):482-9.

### Kits

Also provided are kits comprising any of the compositions described herein. In certain embodiments, the kit comprises a pharmaceutical composition comprising one or more matrix-modifying enzymes. In certain embodiments, the one or more matrix-modifying enzymes have proteolytic activity against a collagen, a proteoglycan, or a combination thereof. In some embodiments the one or more matrix-modifying enzymes comprise a collagenase, wherein the collagenase has proteolytic activity against collagen I, collagen II, collagen III, collagen IV, or any combination thereof. In some embodiments, the collagenase is collagenase P, MMP8, or a combination thereof. In some embodiments, the collagenase is collagenase P from Clostridium histolyticum. In some embodiments, the MMP8 is full-length MMP8. In certain embodiments, the one or more matrix-modifying enzymes are encapsulated in a hydrogel. In some embodiments, the hydrogel comprises alginate. In certain embodiments, the one or more matrix-modifying enzymes are encapsulated in a liposome or conjugated to the surface of a liposome. In some embodiments, the liposome comprises a phospholipid. Exemplary phospholipids include, without limitation, phosphatidylcholine and phosphatidylethanolamine. In some embodiments, the liposome further comprises cholesterol. In some embodiments, the liposome further comprises polyethylene glycol.

Kits may comprise one or more containers of the compositions described herein. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device.

The kit may also provide a delivery device pre-filled with the pharmaceutical composition comprising one or more matrix-modifying enzymes. Formulations suitable for local administration to a CEP are of particular interest, and in such embodiments the kit may include a syringe (e.g., with an epidural needle) or other devices to accomplish such administration.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-80 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A method of treating intervertebral disc degeneration in a subject in need thereof, the method comprising administering a therapeutically effective amount of one or more matrix-modifying enzymes locally to a cartilage endplate (CEP).
2. The method of aspect 1, wherein treatment increases permeability of the CEP and intervertebral disc nutrient transport.
3. The method of aspect 1 or 2, wherein the one or more matrix-modifying enzymes have proteolytic activity against a collagen, a proteoglycan, or a combination thereof.
4. The method of aspect 3, wherein the one or more matrix-modifying enzymes comprise a collagenase.
5. The method of aspect 4, wherein the collagenase has proteolytic activity against collagen I, collagen II, collagen III, collagen IV, or any combination thereof.
6. The method of aspect 5, wherein the collagenase is collagenase P, matrix metalloproteinase 8 (MMP8), or a combination thereof.
7. The method of any one of aspects 4-6, wherein the MMP8 is full-length MMP8.
8. The method of any one of aspects 1-7, wherein the one or more matrix-modifying enzymes are encapsulated in a hydrogel.
9. The method of aspect 8, wherein the hydrogel comprises alginate.
10. The method of aspect 9, wherein the alginate concentration in the hydrogel ranges from 0.5 to 10 percentage by weight (wt %).
11. The method of aspect 10, wherein the alginate concentration in the hydrogel is about 1 wt %.
12. The method of any one of aspects 9-11, wherein the alginate is at least partially oxidized.
13. The method of aspect 12, wherein about 4% to about 10% of the alginate is oxidized.
14. The method of any one of aspects 8-13, wherein the hydrogel sustains delivery of the matrix-modifying enzyme for at least 2 days.
15. The method of any one of aspects 1-7, wherein the one or more matrix-modifying enzymes are encapsulated in a liposome or conjugated to the surface of a liposome.
16. The method of aspect 15, wherein the liposome comprises a phospholipid.
17. The method of aspect 16, wherein the phospholipid is phosphatidylcholine or phosphatidylethanolamine.
18. The method of any one of aspects 15-17, wherein the liposome further comprises cholesterol.
19. The method of any one of aspects 15-18, wherein the liposome further comprises polyethylene glycol.
20. The method of any one of aspects 1-19, wherein the one or more matrix-modifying enzymes are administered in a volume sufficiently small such that intradiscal pressure is not substantially increased.
21. The method of aspect 20, wherein the one or more matrix-modifying enzymes are administered in a volume of no more than 50 μL.
22. The method of any one of aspects 1-21, wherein the one or more matrix-modifying enzymes are administered into or adjacent to the CEP.
23. The method of any one of aspects 1-22, wherein the one or more matrix-modifying enzymes are administered with an epidural needle.
24. The method of aspect 23, wherein the epidural needle has a curved end such that the the matrix-modifying enzyme exits the epidural needle laterally at about a 45-degree angle.
25. The method of any one of aspects 1-24, further comprising using medical imaging to guide said administering the one or more matrix-modifying enzymes locally to the CEP or for selection of which intervertebral discs may benefit from treatment with the one or more matrix-modifying enzymes.
26. The method of aspect 25, wherein said administering the one or more matrix-modifying enzymes locally to the CEP is performed with fluoroscopic guidance, computed tomographic (CT) guidance, combined CT-fluoroscopic guidance, or ultrasound guidance.
27. The method of any one of aspects 1-26, wherein the one or more matrix-modifying enzymes are administered locally to the CEP without exposing the nucleus pulposus to the one or more matrix-modifying enzymes.
28. The method of any one of aspects 1-27, further comprising administering a growth factor.
29. The method of any one of aspects 1-28, further comprising administering an analgesic agent or an anti-inflammatory agent.
30. The method of any one of aspects 1-29, wherein multiple cycles of treatment are administered to the subject.
31. A composition comprising one or more matrix-modifying enzymes for use in a method of treating intervertebral disc degeneration.
32. The composition of aspect 31, further comprising a pharmaceutically acceptable excipient.
33. The composition of aspect 31 or 32, wherein the one or more matrix-modifying enzymes comprise a collagenase.
34. The composition of aspect 33, wherein the collagenase has proteolytic activity against collagen I, collagen II, collagen III, collagen IV, or any combination thereof.
35. The composition of aspect 34, wherein the collagenase is collagenase P, matrix metalloproteinase 8 (MMP8), or a combination thereof.
36. The composition of any one of aspects 33-35, wherein the MMP8 is full-length MMP8.
37. The composition of any one of aspects 31-36, wherein the one or more matrix-modifying enzymes are encapsulated in a hydrogel.
38. The composition of aspect 37, wherein the hydrogel comprises alginate.
39. The composition of aspect 38, wherein the alginate concentration in the hydrogel ranges from 0.5 to 10 percentage by weight (wt %).
40. The composition of aspect 39, wherein the alginate concentration in the hydrogel is about 1 wt %.
41. The composition of any one of aspects 38-40, wherein the alginate is at least partially oxidized.
42. The composition of aspect 41, wherein about 4% to about 10% of the alginate is oxidized.
43. The composition of any one of aspects 37-42, wherein the hydrogel sustains delivery of the matrix-modifying enzyme for at least 2 days.
44. The composition of any one of aspects 31-36, wherein the one or more matrix-modifying enzymes are encapsulated in a liposome or conjugated to the surface of a liposome.
45. The composition of aspect 44, wherein the liposome comprises a phospholipid.
46. The composition of aspect 45, wherein the phospholipid is phosphatidylcholine or phosphatidylethanolamine.
47. The composition of any one of aspects 44-46, wherein the liposome further comprises cholesterol.
48. The composition of any one of aspects 44-47, wherein the liposome further comprises polyethylene glycol.
49. The composition of any one of aspects 31-48, further comprising a growth factor.
50. The composition of any one of aspects 31-49, further comprising an analgesic agent or an anti-inflammatory agent.
51. A method of increasing intervertebral disc solute transport in a subject in need thereof, the method comprising administering an effective amount of one or more matrix-modifying enzymes locally to a cartilage endplate (CEP).
52. The method of aspect 51, wherein said administering increases permeability of the CEP and intervertebral disc nutrient transport.
53. The method of aspect 51 or 52, wherein the one or more matrix-modifying enzymes have proteolytic activity against a collagen, a proteoglycan, or a combination thereof.
54. The method of aspect 53, wherein the one or more matrix-modifying enzymes comprise a collagenase.
55. The method of aspect 54, wherein the collagenase has proteolytic activity against collagen I, collagen II, collagen III, collagen IV, or any combination thereof.
56. The method of aspect 55, wherein the collagenase is collagenase P, matrix metalloproteinase 8 (MMP8), or a combination thereof.
57. The method of any one of aspects 54-56, wherein the MMP8 is full-length MMP8.
58. The method of any one of aspects 51-57, wherein the one or more matrix-modifying enzymes are encapsulated in a hydrogel.
59. The method of aspect 58, wherein the hydrogel comprises alginate.
60. The method of aspect 59, wherein the alginate concentration in the hydrogel ranges from 0.5 to 10 percentage by weight (wt %).
61. The method of aspect 60, wherein the alginate concentration in the hydrogel is about 1 wt %.
62. The method of any one of aspects 59-61, wherein the alginate is at least partially oxidized.
63. The method of aspect 62, wherein about 4% to about 10% of the alginate is oxidized.
64. The method of any one of aspects 58-63, wherein the hydrogel sustains delivery of the matrix-modifying enzyme for at least 2 days.
65. The method of any one of aspects 51-57, wherein the one or more matrix-modifying enzymes are encapsulated in a liposome or conjugated to the surface of a liposome.
66. The method of aspect 65, wherein the liposome comprises a phospholipid.
67. The method of aspect 66, wherein the phospholipid is phosphatidylcholine or phosphatidylethanolamine.
68. The method of any one of aspects 65-67, wherein the liposome further comprises cholesterol.
69. The method of any one of aspects 65-68, wherein the liposome further comprises polyethylene glycol.
70. The method of any one of aspects 51-69 wherein the one or more matrix-modifying enzymes are administered in a volume sufficiently small such that intradiscal pressure is not substantially increased.
71. The method of aspect 70, wherein the one or more matrix-modifying enzymes are administered in a volume of no more than 50 μL.
72. The method of any one of aspects 51-71, wherein the one or more matrix-modifying enzymes are administered into or adjacent to the CEP.
73. The method of any one of aspects 51-72, wherein the one or more matrix-modifying enzymes are administered with an epidural needle.
74. The method of aspect 73, wherein the epidural needle has a curved end such that the the one or more matrix-modifying enzymes exit the epidural needle laterally at about a 45-degree angle.
75. The method of any one of aspects 51-74, further comprising using medical imaging to guide said administering the one or more matrix-modifying enzymes locally to the CEP or selection of which intervertebral discs may benefit from treatment with the one or more matrix-modifying enzymes.
76. The method of aspect 75, wherein said administering the one or more matrix-modifying enzymes locally to the CEP is performed with fluoroscopic guidance, computed tomographic (CT) guidance, combined CT-fluoroscopic guidance, or ultrasound guidance.
77. The method of any one of aspects 51-76, wherein the one or more matrix-modifying enzymes are administered locally to the CEP without exposing the nucleus pulposus to the one or more matrix-modifying enzymes.
78. The method of any one of aspects 51-77, further comprising administering a growth factor.
79. The method of any one of aspects 51-78, further comprising administering an analgesic agent or an anti-inflammatory agent.
80. The method of any one of aspects 51-79, wherein prior to said administering the one or more matrix-modifying enzymes locally to the CEP, the method further comprises detecting if intervertebral disc solute transport is impeded in the CEP to determine if the subject will benefit from the treatment.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventors to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### Intradiscal Treatment of the Cartilage Endplate for Improving Solute Transport and Disc Nutrition

Proper disc nutrition involves nutrient and metabolite exchange between the nucleus pulposus (NP) cells and vertebral capillaries (Maroudas et al., 1975; Nachemson et al., 1970; Ohshima et al., 1989; Urban et al., 1977), and several factors can impair the normal patterns of solute exchange. For example, nutrients entering the disc and exiting metabolites must pass through the cartilage endplates (CEP), and solute passage could be slowed or blocked by changes to the composition of the CEP matrix, including dehydration (Antoniou et al., 1996), mineralization (Wong et al., 2019, Benneker 2005; Grant 2016) and fibrosis (Antoniou et al., 1996; Wong et al., 2019). For example, we found that CEPs with higher amounts of collagen, sulfated glycosaminoglycan (sGAG), and mineral hindered nutrient diffusion, thereby impairing NP cell survival and function inside diffusion chambers (Wong et al., 2019). Moreover, those same deficits in CEP composition were also associated with significantly worse disc degeneration in low back pain patients (Bonnheim et al., 2022).

Motivated by those findings, we explored a matrix modification strategy for improving disc nutrition that involves enzymatically reducing the amounts of collagen and aggrecan in the impermeable and fibrotic CEPs (Dolor et al., 2019). Treatment of human cadaveric CEPs with recombinant human MMP-8 enzyme, which is one of the matrix metalloproteinases that has a high specificity for type II collagen and aggrecan, reduced sGAG content in a dose-dependent manner. Importantly, reductions in CEP matrix content with MMP-8 treatment improved the NP cell viability inside diffusion chambers, indicating improved CEP permeability to nutrients (Dolor et al., 2019). Although the diffusion chambers represent a useful model system for studying the effects of CEP permeability enhancement, the chambers do not mimic the complex microenvironment or loading of an intact intervertebral disc, and thus, the effect of CEP permeability enhancement on solute transport into whole, intact discs remains unclear. The objectives of this study were to: 1) develop a model for the controlled, intradiscal delivery of a matrix-modifying enzyme to the CEP; and 2) measure solute transport into whole human and bovine discs following intradiscal treatment of the CEP with a matrix-modifying enzyme.

### Methods

### Bovine Tissues:

Nine intact bovine coccygeal motion segments were harvested from three oxtails (3 segments per oxtail) acquired within 2 hr of slaughter from a local abattoir. After harvesting the motion segments, a 17G Crawford needle with a plasma-mediated coblation wand (ArthroCare 2000) was inserted through the posteriolateral annulus directed toward one CEP in each disc, the wand was energized on a low-power setting (2 W) for 10 sec as the needle was advanced to the distal annulus, and the coblated tissue was debrided with PBS. This coblation step was necessary for overcoming the high back pressure in the otherwise healthy bovine discs. Following coblation, the wand was removed with the needle in place, and the motion segments were randomly assigned to three groups (n=3 motion segments/group; Table 2): collagenase—0.15 U in 50 μL of slow-release alginate carrier; control buffer—HBSS in 50 μL of alginate carrier; or sham—needle insertion only.

### Human Tissues:

Five human cadaveric lumbar spines (age range: 38-66 years old; mean age: 56±10 years) were obtained from donors with no medical history of musculoskeletal disorders (UCSF Willed Body Program). From these spines, ten intact motion segments (2 motion segments per spine) were harvested and randomly assigned to two groups (n=5 motion segments/group; Table 1); collagenase—0.15 U in 50 μL of alginate carrier; or control buffer—HBSS in 50 μL of alginate carrier).

### Alginate Preparation:

For alginate purification, sodium alginate (10 g, Protanal LF 20/40) was dissolved in 1000 ml ultrapure deionized water (diH₂O), dialyzed against diH₂O (MWCO 3500; Spectrum Laboratories Inc.) for 3 days, treated with activated charcoal (0.5 mg/100 ml, 50-200 mesh, Fisher) for 30 min, filtered (0.22 μm filter) and lyophilized.

Oxidized alginates (OA) were prepared by reacting sodium alginate (Protanal LF 20/40, 196,000 g/mol, FMC Biopolymer) with sodium periodate (Sigma) using a previously described method (Jeon et al., 2017). Briefly, sodium alginate (10 g) was dissolved in ultrapure deionized water (diH₂O, 900 ml) overnight. Sodium periodate (543 and 1085 mg) was dissolved in 100 ml diH₂O and added to separate alginate solutions to achieve different degrees of theoretical alginate oxidation (5 and 10%, respectively) under stirring in the dark at room temperature for 24 hours. The OAs were purified by dialysis against diH₂O (MWCO 3500; Spectrum Laboratories Inc.) for 3 days, treated with activated charcoal (0.5 mg/100 ml, 50-200 mesh, Fisher) for 30 min, filtered (0.22 μm filter) and lyophilized. The actual oxidation (4.2±0.5 and 8.5±0.3%) was determined by using the Amplite™ Colorimetric Aldehyde Quantitation Kit (AAT Bioquest Inc.) according to the manufacturer's instructions.

### Intradiscal CEP Treatment:

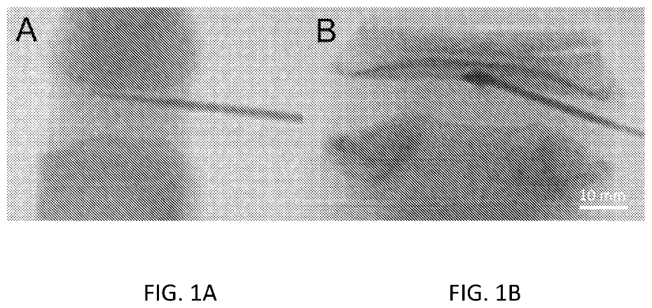

Enzymatic treatment or control buffer was delivered to the CEP under fluoroscopic guidance. Briefly, a 17G Tuohy needle (Medline, item: PAIN8001) was inserted through the posterolateral annulus fibrosus and advanced to the central region of the superior CEP in each disc (FIG. 1). After needle placement, 50 μL of either collagenase or control buffer that was suspended in a slow-release alginate carrier was injected to the CEP, and then fibrin sealant (TISSEEL, Baxter International Inc., CA, USA) was applied to the annulus during needle retraction. All motion segments were then incubated in protease inhibitor cocktail (Sigma-Aldrich, catalog number P2714) for 18 hr at 37 °C).

### Transport Experiment:

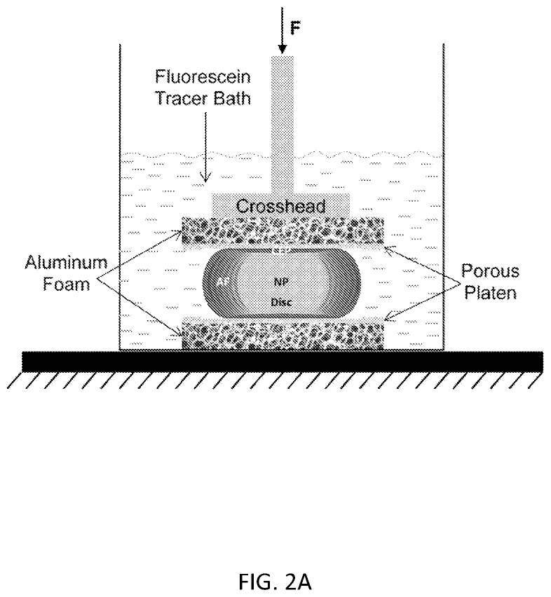

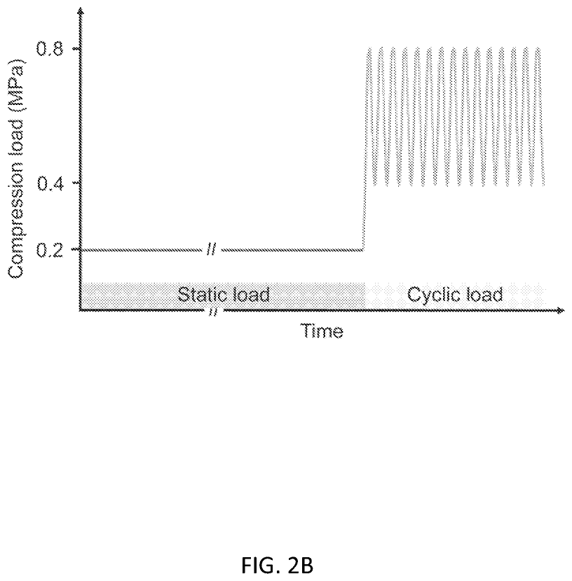

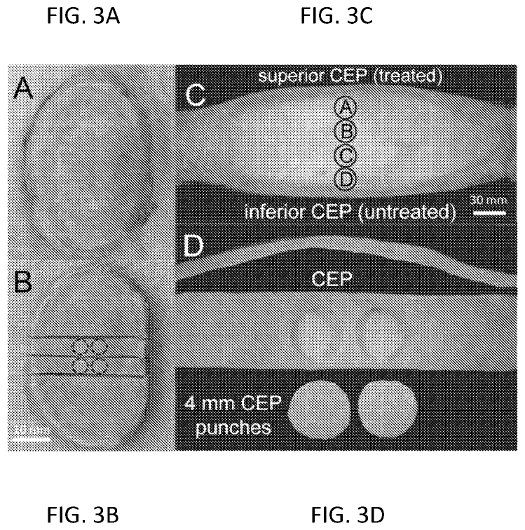

A custom setup was used to evaluate the effects of CEP permeability enhancement on solute transport into the disc (FIG. 2). To eliminate any potentially confounding effects of differences in bony endplate structure within and between species, the inferior and superior bony endplates were carefully removed from the motion segments with a burring tool (FIG. 3A). Several x-ray images (Faxitron Cabinet X-ray Systems model 43855A) were taken during the bone removal process to ensure the complete removal of all bone tissues and to calculate the cross-sectional area of the isolated discs (ImageJ, open-source software, Schneider, C. A., 2012).

The isolated discs were then sandwiched between porous stainless-steel platens (0.8 mm hole diameter, 1.8 mm thickness and 17.9% hole coverage) and open-cell porous aluminum foam blocks with similar porosity and stiffness of vertebral trabecular bone (Duocel® aluminum foam disc, 7-9% relative density, ERG Aerospace Corp, Oakland, CA) while submerged in 200 ml of fluorescein tracer solution (376 Da; 0.1 mg/mL in PBS) (FIG. 2). A servo-hydraulic load frame (MTS Bionix 858; MTS Systems Corp., Eden Prairie, MN) was used to apply loads resulting in physiologic disc pressures. Specifically, we applied 6 hr of static compression loads that represent the lower end of intradiscal pressure range measured during various static postures (0.2 MPa) (Nachemson 1966; Wilke, et al. 1999), followed by 1.6 hr of cyclic compressive loads resulting in a pressure range (0.4-0.8 MPa) and frequency (0.2 Hz) that is similar to those measured in the disc during walking (Wilke, et al., 1999).

### NP and CEP Tissue Sampling:

Immediately after the transport experiments, discs were snap frozen in liquid nitrogen to prevent any further fluorescein dispersion. Next, two 5-mm-thick-coronal slabs were cut with a low-speed diamond saw (IsoMet, Buehler, IL) (FIG. 3B). From each slab, three NP samples were collected using a 2-mm biopsy punch at four superior-inferior locations (FIG. 3C). The superior and inferior CEP layers were then carefully removed from the discs and trimmed of any residual NP tissue (FIG. 3D). Two CEP samples were collected from each intact CEP using a 4-mm biopsy punch (FIG. 3D). All NP and CEP samples were massed on microbalance, dried by lyophilization, and completely digested in papain for subsequent assays.

### Fluorescein Concentration:

NP tissue digests were assayed for fluorescence on a SpectraMax iD5 microplate reader (Molecular Devices, San Jose, CA), and the fluorescence readings (ex. 485 nm, em. 525 nm) referenced to a standard curve of known fluorescein concentrations. Fluorescein concentrations inside the NP samples were calculated based on the measured water content from lyophilization and were compared between site-matched locations in the discs having collagenase-treated vs. buffer-treated CEPs.

### CEP Biochemical Composition:

CEP tissue digests were assayed for sGAG content using a dimethylmethylene blue assay (DMMB) (Farndale R W 1986; Sampson 2019). The digest absorbance was measured with a microplate reader at 595 nm and referenced to a standard curve of known chondroitin sulfate concentrations.

Aliquots of CEP digests were also hydrolyzed in 6 N HCL for 18 h at 110 °C. Neutralized acid hydrolysates were then assayed for total collagen content using a chloramine-T colorimetric assay (Fields A. 2014), referencing the absorbance measurements to a standard curve of known hydroxyproline concentrations.

The total sGAG and collagen contents in the CEP tissues were used to evaluate the effect of the enzymatic treatment on the CEP matrix composition.

### Whole Disc Biomechanical Properties:

The axial force, crosshead displacement and time data were collected during the transport experiment and processed with a custom script (MATLAB, Mathworks; Natwick, MA) that calculates the disc modulus and the total creep strain. Disc modulus was considered as the slope of the stress-strain curve at maximum value of the stress in the linear part of the static compression phase. The total creep strain was considered as the ratio between total displacement at the end of the static compression phase and the initial height of the disc.

### Statistics:

Mixed effects models accounting for multiple measurements per subject via random intercept were used to estimate the least-squares mean fluorescein concentration at each NP location and the least-squares mean sGAG and collagen content in the CEP, and Tukey HSD post-hoc tests were used to test for differences between groups. One-way ANOVA was used to compare the whole-disc compressive creep strain and modulus between groups. Statistical analyses were performed in JMP Pro 15; p<0.05 was considered statistically significant.

### Results

### Effects of Enzymatic Treatment on Solute Transport

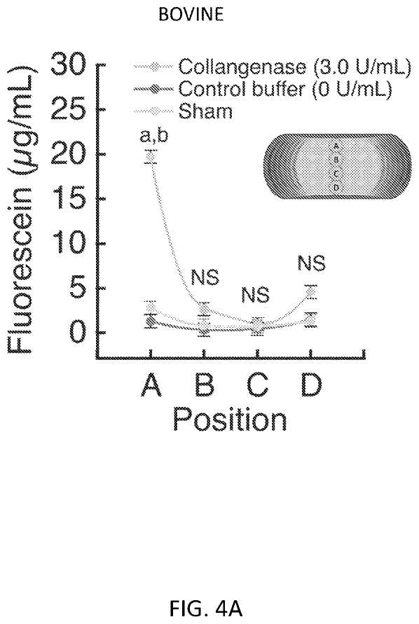

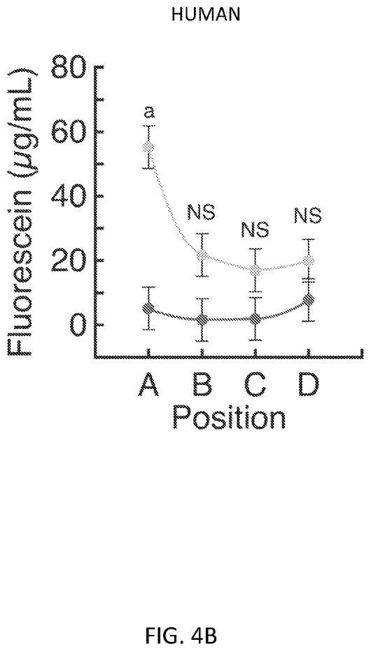

Intradiscal enzymatic treatment of the CEP significantly improved solute transport into the bovine coccygeal discs (FIG. 4A). Specifically, in the NP near to the superior treated CEP, fluorescein tracer concentration was 14-fold higher in the discs that had CEPs treated with collagenase than in the discs that had CEPs treated with control buffer (19.72±5.17 μg/mL vs. 1.41±1.11 μg/mL; p<0.0001) and 7-fold higher than discs with untreated CEPs (2.81±1.79 μg/mL; sham). In discs with buffer-treated and sham CEPs, fluorescein concentrations were generally highest near the CEPs, at positions A (buffer: 1.41±1.11; sham: 2.81±1.79 μg/mL) and D (buffer: 1.48±1.66; sham: 1.35±0.52 μg/mL), and lowest in the center of the discs, at positions B (buffer: 0.33±0.14; sham: 0.81±0.34 μg/mL) and C (buffer: 0.43±0.27; sham: 0.71±0.25 μg/mL).

Similarly, intradiscal enzymatic treatment of the CEP improved solute transport into human lumbar discs (FIG. 4B). The fluorescein concentration in the NP near to the superior treated CEP, at position A, was 10.8-fold higher inside the discs that had CEPs treated with collagenase than in the discs that had CEPs treated with the control buffer (55.27±31.11 μg/mL vs. 5.14±3.54 μg/mL; p<0.0001). In the discs with buffer-treated CEPs, fluorescein concentration varied with position; concentrations were generally highest adjacent to the CEPs, positions A (5.14±3.54 μg/mL) and D (7.14±2.26 μg/mL), and lowest in the center of the discs, at positions B (0.58±0.18 μg/mL) and C (0.62±0.19 μg/mL).

### Effects of Enzymatic Treatment on CEP Matrix Composition

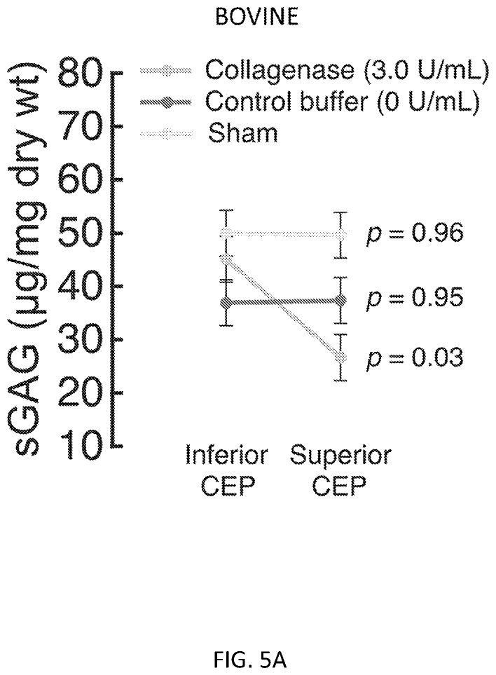

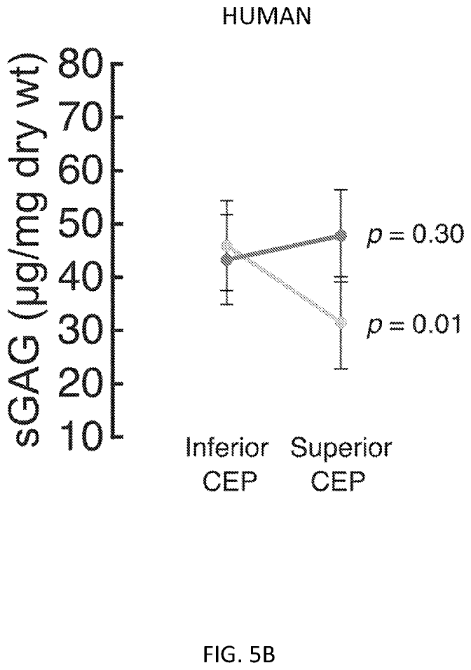

Intradiscal enzymatic treatment significantly reduced the amount of solute-blocking matrix constituents in both bovine and human CEPs. Specifically, collagenase treatment reduced sGAG content, with treated bovine CEPs showing, on average, 40% lower sGAG content than their paired untreated CEPs (p=0.03; FIG. 5A). The CEPs that were untreated as well as those that were treated with the control buffer had similar sGAG contents. Likewise, the same enzyme dose delivered intradiscally to human CEPs significantly reduced sGAG content, with the superior CEPs showing 33.5% lower sGAG content, on average, than their paired inferior CEPs (p=0.01; FIG. 5B).

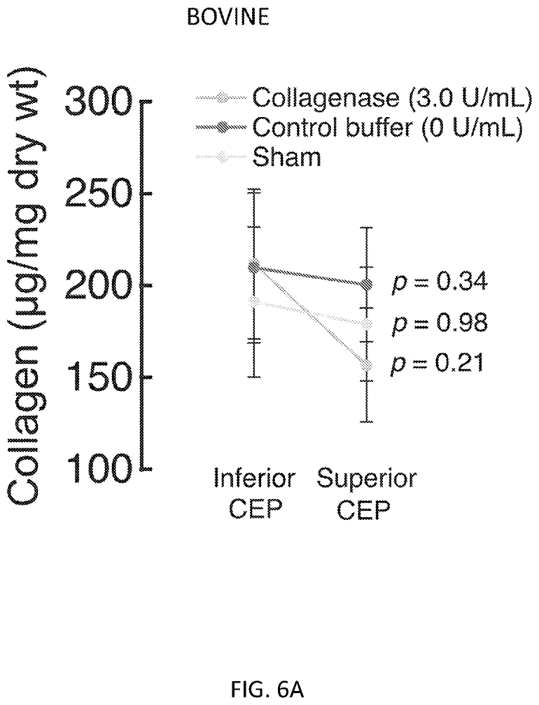

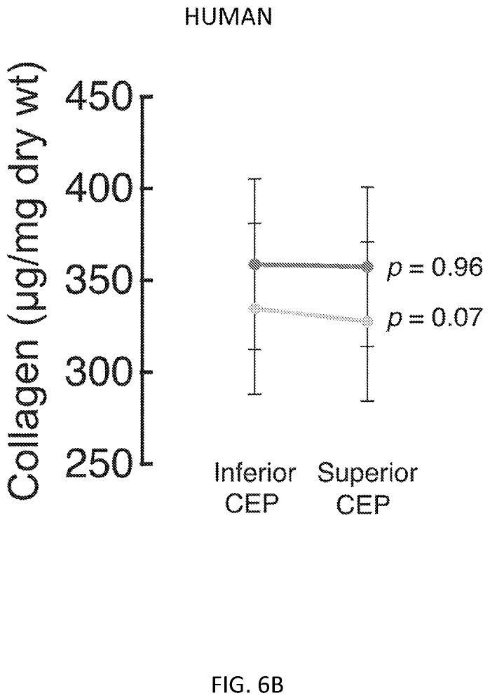

Collagenase treatment tended to associate with lower collagen content in the treated CEPs, but the differences were not statistically significant. Specifically, mean collagen content was lower in the superior treated CEPs (156.79±84.55 μg/mg dry wt) than their paired inferior CEPs (233.39±85.73 μg/mg dry wt; p=0.21; FIG. 6A), but the difference did not reach statistical significance. In the human discs that underwent collagenase treatment, collagen content in the treated superior CEPs (327.75±188.65 μg/mg dry wt) tended to be lower than their paired untreated CEPs (334.7186±208.01 μg/mg dry wt; p=0.07; FIG. 6B).

### Effects of Enzymatic Treatment on NP Tissue Composition

To understand how the enzymatic treatment delivered to the CEP affected the composition of the NP tissue, we measured the sGAG content in the human NP tissue samples that were assayed for fluorescein (Table 3). Collagenase treatment had a small, localized effect on the sGAG content of the adjacent NP tissue. Specifically, mean sGAG of NP samples adjacent to the collagenase-treated CEP at position A was 15.2% lower than the NP tissue samples adjacent to the untreated CEP at position D in the same discs (p=0.027) and 13.8% lower than the tissue NP samples at position B in the same discs (p=0.026). Comparisons of mean sGAG between the NP tissue samples at the other positions were not significant.

### Effects of Enzymatic Treatment on Whole-Disc Biomechanical Behavior

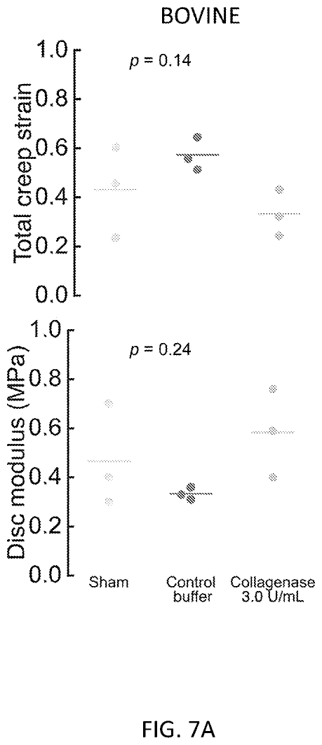

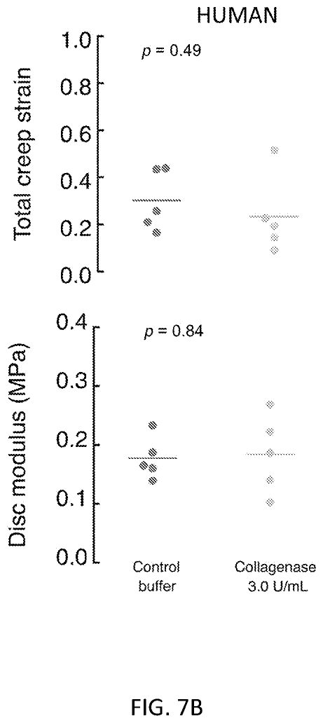

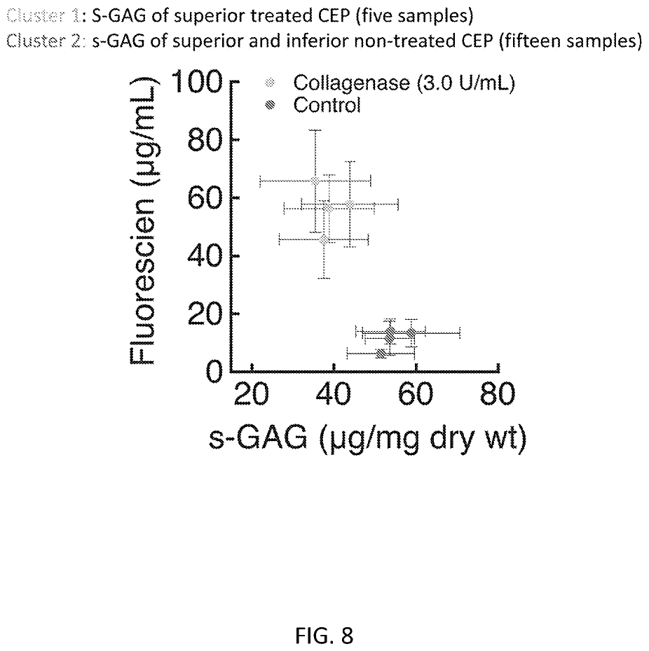

To determine the biomechanical effects of CEP matrix modification at the whole-disc level, we compared the compressive modulus and total creep strain between groups by ANOVA. In the bovine discs, total creep strain and compressive modulus were similar between discs with the collagenase-treated CEPs, buffer-treated CEPs, and untreated CEPs (FIG. 7A). Likewise, in the human discs, total creep strain and compressive modulus were similar between discs with collagenase-treated and buffer-treated CEPs (FIG. 7B).

### Discussion

Here we investigated intradiscal treatment to modify the matrix of the CEP to enhance the transport of small solutes into whole intervertebral discs. Collagenase enzyme was loaded into a degradable hydrogel carrier and then delivered via needle to the superior CEP under fluoroscopic guidance. Results show that collagenase treatment (0.15 U) significantly reduced the amount of sGAG in the treated CEP: enzymatic treatment of human lumbar and bovine coccygeal CEPs reduced sGAG by 33.5% and 40%, respectively (FIG. 5). Enzymatic treatment also led to reductions in collagen content, but the treatment effects were heterogeneous, and the differences in CEP collagen content between treated and untreated CEPs of the same disc were not statistically significant. Importantly, enzymatic treatment of the CEP coincided with greater transport of 376-Da fluorescein into the nucleus pulposus under static and cyclic compressive loads. Specifically, NP tissue adjacent to collagenase-treated bovine and human CEPs had 10.8-fold (bovine) and 14-fold (human) higher fluorescein concentrations compared to the NP tissue adjacent to buffer-treated CEP after loading (FIG. 4). The similar pattern of the findings in bovine discs with relatively healthy CEP and NP tissues and human discs with more degenerated CEP and NP tissue lends confidence in the generalizability of the treatment mechanism and the overall magnitude of the treatment effect. In our previous study of human CEPs, we observed that ex situ treatment of CEPs with truncated human recombinant MMP-8 reduced sGAG content in a dose-dependent manner, increased fluorescein uptake into unloaded CEPs, and enhanced nutrient transport and NP cell viability (Dolor et al., 2019) Our new findings demonstrate for the first time the concept of CEP matrix modification to whole, compressively loaded discs, and taken together, these findings support the potential for matrix modification of the CEP to improve the transport of small solutes in the size range of essential nutrients, e.g. glucose (180 Da).

The goal of CEP matrix modification is to reduce solute-blocking matrix constituents, improve the CEP's permeability to nutrients, and enhance disc cell viability/function (Dolor et al., 2019; Wong et al., 2019). In our previous study, reducing the amount of sGAG in the CEP by 6-22% coincided with increases in passive fluorescein uptake by 16-24% (Dolor et al., 2019). In vivo, the disc is subjected to a combination of static and cyclic loads, which improves solute transport through the CEP by inducing convection (Sampson et al., 2019). The effects of fluid flow on solute convection may explain why a similar enzyme dose used in the current study produced a larger increase in fluorescein transport through the CEP than observed with passive diffusion.

The CEP is rich in type II collagen (Moore, 2006; Nosikova et al., 2012; Schollmeier et al., 2000; Roberts S, 1991) and aggrecan. We used a commercial form of collagenase P from Clostridium histolyticum (C. histolyticum) for matrix modification, as this enzyme can break down the peptide bonds in four types of collagens (I, II, III, and IV) (Alipour et al., 2016) and has proteolytic activity on proteoglycans. Matrix metalloproteinases (MMPs) also have a high specificity for type II collagen and aggrecan (Dolor et al., 2019; Hideaki and Kashiwagi 2002). In the current study, we treated both human and bovine CEPs, so we chose a generic collagenase enzyme rather than a specific human recombinant MMP. Nevertheless, the effects of collagenase P on human CEP composition appeared similar to those observed with truncated human MMP-8 (Dolor et al., 2019), and together our findings suggest that both enzymes reduce sGAG content in the CEP and have more subtle effects on the collagen. Although collagen content in the collagenase-treated CEPs was typically lower than buffer-treated CEPs, these differences were not statistically significant (FIG. 6). One possible explanation is that cleaved collagen fragments remained intertwined in the CEP matrix, and greater exposure times are required for the removal of these fragments (Jiang et al., 2020).

Targeted delivery of matrix-modifying enzymes to the CEP presents several technical challenges. We chose a 50 μL volume of injectate based on preliminary intradiscal pressure testing; injecting larger volumes increased the back pressure and made it difficult to prevent leakage. Choice of the hydrogel carrier was made to control the release of the enzymatic treatment (Supplementary Materials), and intradiscal delivery was performed using a 17G Tuohy epidural needle with a curved end at the needle's tip that allows the hydrogel to exit laterally at a 45-degree angle. Combined with the oblique trajectory of the needle, it was possible to deliver the treatment adjacent to targeted CEP (FIG. 1). These factors aided in localizing the enzyme and minimizing the effects on the nucleus pulposus tissue; however, we found that NP tissues adjacent to these enzyme-treated CEPs had 15% lower sGAG content compared to the NP tissues adjacent to the untreated CEP of the same disc (Table 3). This suggests that other strategies may be needed to further reduce off-target treatment effects. For example, attaching the enzyme to large liposomal nanoparticles may limit enzyme mobility and help reduce unwanted enzyme migration in the NP (Dolor et al., 2019).

Proper CEP function requires balancing conflicting biotransport and biomechanical demands, and thus, successful matrix modification strategies for improving biotransport must be sensitive to any biomechanical effects. Specifically, excessive cleavage and removal of collagen fibrils or proteoglycans in the CEP could inadvertently lower disc modulus and risk clinically significant disc injury. The collagen network helps resist transverse CEP stretching that arises during disc compression (Fields et al., 2010; DeLucca et al., 2016; Fields et al., 2014), and proteoglycans in the CEP are believed to help prevent the loss of proteoglycan aggregates from the nucleus pulposus (Roberts et al., 1996). We found that discs with enzyme-treated CEPs had similar whole-disc modulus and total creep strain as buffer-treated discs (FIG. 7), which suggests that the modest dose used here was sufficient to improve solute transport without impacting whole-disc behavior under static and cyclic compressive loading. Additional mechanical testing with more complex loading protocols is needed to further understand the biomechanical effects of CEP matrix modification.

### CONCLUSION

We found that intradiscal delivery of a matrix-modifying enzyme to the CEP significantly reduced the amount of solute-blocking matrix constituents, specifically the amount of sGAG. Treating the CEP in this manner dramatically enhanced small solute transport into the disc. This is an important step towards in vivo pre-clinical studies.

### REFERENCES

Alipour, H., Raz, A., Zakeri, S., Dinparast Djadid, N., 2016. Therapeutic applications of collagenase (metalloproteases): A review. Asian Pacific Journal of Tropical Biomedicine. doi.org/10.1016/j.apjtb.2016.07.017
Bae, W. C., Masuda, K., 2011. Emerging Technologies for Molecular Therapy for Intervertebral Disk Degeneration. Orthopedic Clinics of North America. doi.org/10.1016/j.ocl.2011.07.004
Bonnheim, N. B., Wang, L., Lazar, A. A., Zhou, J., Chachad, R., Sollmann, N., Guo, X., Iriondo, C., O'Neill, C., Lotz, J. C., Link, T. M., Krug, R., Fields, A. J., 2022. The contributions of cartilage endplate composition and vertebral bone marrow fat to intervertebral disc degeneration in patients with chronic low back pain. European Spine Journal. doi.org/10.1007/s00586-022-07206-x
Chou, D., Samartzis, D., Bellabarba, C., Patel, A., Luk, K. D. K., Kisser, J. M. S., Skelly, A. C., 2011. Degenerative magnetic resonance imaging changes in patients with chronic low back pain: A systematic review. Spine (Phila Pa 1976). doi.org/10.1097/BRS.0b013e31822ef700
Clin Invest, A. J., Antoniou, J., Steffen, T., Nelson, F., Winterbottom, N., Hollander, A. P., Poole, R. A., Aebi, M., Alini, M., 1996a. The Human Lumbar Intervertebral Disc Evidence for Changes in the Biosynthesis and Denaturation of the Extracellular Matrix with Growth, Maturation, Ageing, and Degeneration.
Clin Invest, A. J., Antoniou, J., Steffen, T., Nelson, F., Winterbottom, N., Hollander, A. P., Poole, R. A., Aebi, M., Alini, M., 1996b. The Human Lumbar Intervertebral Disc Evidence for Changes in the Biosynthesis and Denaturation of the Extracellular Matrix with Growth, Maturation, Ageing, and Degeneration.
DeLucca, J. F., Cortes, D. H., Jacobs, N. T., Vresilovic, E. J., Duncan, R. L., Elliott, D. M., 2016. Human cartilage endplate permeability varies with degeneration and intervertebral disc site. Journal of Biomechanics 49, 550-557. doi.org/10.1016/j.jbiomech.2016.01.007
Dolor, A., Sampson, S. L., Lazar, A. A., Lotz, J. C., Szoka, F. C., Fields, A. J., 2019. Matrix modification for enhancing the transport properties of the human cartilage endplate to improve disc nutrition. PLoS ONE 14. doi.org/10.1371/journal.pone.0215218
Fernandez-Moure, J., Moore, C. A., Kim, K., Karim, A., Smith, K., Barbosa, Z., van Eps, J., Rameshwar, P., Weiner, B., 2018. Novel therapeutic strategies for degenerative disc disease: Review of cell biology and intervertebral disc cell therapy. SAGE Open Medicine 6, 205031211876167. doi.org/10.1177/2050312118761674
Fields, A. J., Ballatori, A., Liebenberg, E. C., Lotz, J. C., 2018. Contribution of the Endplates to Disc Degeneration. Current Molecular Biology Reports 4, 151-160. doi.org/10.1007/s40610-018-0105-y
Fields, A. J., Lee, G. L., Keaveny, T. M., 2010. Mechanisms of initial endplate failure in the human vertebral body. Journal of Biomechanics 43, 3126-3131. doi.org/10.1016/j.jbiomech.2010.08.002
Fields, A. J., Rodriguez, D., Gary, K. N., Liebenberg, E. C., Lotz, J. C., 2014. Influence of biochemical composition on endplate cartilage tensile properties in the human lumbar spine. Journal of Orthopaedic Research 32, 245-252. doi.org/10.1002/jor.22516
Gawri, R., Antoniou, J., Ouellet, J., Awwad, W., Steffen, T., Roughley, P., Haglund, L., Mwale, F., 2013. Best paper NASS 2013: Link-N can stimulate proteoglycan synthesis in the degenerated human intervertebral discs. European Cells and Materials 26, 107-119. doi.org/10.22203/eCM.v026a08
Haralson lii, R. H., Zuckerman, J. D., n.d. Prevalence, Health Care Expenditures, and Orthopedic Surgery Workforce for Musculoskeletal Conditions.
Nagase, H., Kashiwagi, M. Aggrecanases and cartilage matrix degradation. Arthritis Res Ther 5, 94 (2003). doi.org/10.1186/ar630
Horner H A, Urban J P. 2001 Volvo Award Winner in Basic Science Studies: Effect of nutrient supply on the viability of cells from the nucleus pulposus of the intervertebral disc. Spine (Phila Pa 1976). 2001 Dec. 1; 26(23):2543-9. doi: 10.1097/00007632-200112010-00006. PMID: 11725234
Huang, Y. C., Urban, J. P. G., Luk, K. D. K., 2014. Intervertebral disc regeneration: Do nutrients lead the way?Nature Reviews Rheumatology. doi.org/10.1038/nrrheum.2014.91
Jeon, O., Shin, J. Y., Marks, R., Hopkins, M., Kim, T. H., Park, H. H., Alsberg, E., 2017. Highly elastic and tough interpenetrating polymer network-structured hybrid hydrogels for cyclic mechanical loading-enhanced tissue engineering. Chemistry of Materials 29, 8425-8432. doi.org/10.1021/acs.chemmater.7b02995
Jiang, C. C., Hsieh, C. H., Liao, C. J., Chang, W. H., Liao, W. J., Tsai-Wu, J. J., Chiang, H., 2020. Collagenase treatment of cartilaginous matrix promotes fusion of adjacent cartilage. Regenerative Therapy 15, 97-102. doi.org/10.1016/j.reth.2020.05.006
Lotz, J. C., Fields, A. J., Liebenberg, E. C., 2013. The Role of the Vertebral End Plate in Low Back Pain. Global Spine Journal 3, 153-163. doi.org/10.1055/s-0033-1347298
Maroudas, A., Stockwell, R. A., Nachemson, A., Urban, A. J., 1975. Factors involved in the nutrition of the human lumbar intervertebral disc: cellularity and diffusion of glucose in vitro, J. Anat.
Matta, A., Karim, M. Z., Isenman, D. E., Erwin, W. M., 2017. Molecular Therapy for Degenerative Disc Disease: Clues from Secretome Analysis of the Notochordal Cell-Rich Nucleus Pulposus. Scientific Reports 7. doi.org/10.1038/srep45623
Moore, R. J., 2006. The vertebral endplate: Disc degeneration, disc regeneration, in: European Spine Journal. doi.org/10.1007/s00586-006-0170-4
Nachemson, A. M. D., The Load on Lumbar Disks in Different Positions of the Body. Clinical Orthopaedics and Related Research: March 1966—Volume 45—Issue—p 107-122
Nachemson, A., Lewin, T., Maroudas, A., Freeman, M. A. R., 1970. In vitro diffusion of DYE through the end-plates and the annulus fibrosus of human lumbar inter-vertebral discs. Acta Orthopaedica 41, 589-607. doi.org/10.3109/17453677008991550
Nosikova, Y. S., Santerre, J. P., Grynpas, M., Gibson, G., Kandel, R. A., 2012. Characterization of the annulus fibrosus-vertebral body interface: Identification of new structural features. Journal of Anatomy 221, 577-589. doi.org/10.1111/j.1469-7580.2012.01537.x
Roberts S, Urban J P, Evans H, Eisenstein S M. Transport properties of the human cartilage endplate in relation to its composition and calcification. Spine. 1996 February; 21(4):415-420. DOI: 10.1097/00007632-199602150-00003. PMID: 8658243
Roberts S, M. J. D. V. W. S. A. S., 1991. 1948394. Spine (Phila Pa 1976) 16, 1030-1038.
Rodriguez, A. G., Slichter, C. K., Acosta, F. L., Rodriguez-Soto, A. E., Burghardt, A. J., Majumdar, S., Lotz, J. C., 2011. Human disc nucleus properties and vertebral endplate permeability. Spine (Phila Pa 1976) 36, 512-520. doi.org/10.1097/BRS.0b013e3181f72b94
Sampson, S. L., Sylvia, M., Fields, A. J., 2019. Effects of dynamic loading on solute transport through the human cartilage endplate. Journal of Biomechanics 83, 273-279. doi.org/10.1016/j.jbiomech.2018.12.004
Schollmeier G, Lahr-Eigen R, Lewandrowski K U. Observations on fiber-forming collagens in the anulus fibrosus. Spine (Phila Pa 1976). 2000 Nov. 1; 25(21):2736-41. doi: 10.1097/00007632-200011010-00004. PMID: 11064517
Vassilaki, M., Hurwitz Dc, E. L., 2014. Insights in public health: perspectives on pain in the low back and neck: global burden, epidemiology, and management. Hawai'i journal of medicine & public health: a journal of Asia Pacific Medicine & Public Health, 73(4), 122-126.
Wilke H J, Neef P, Caimi M, Hoogland T, Claes L E. New in vivo measurements of pressures in the intervertebral disc in daily life. Spine (Phila Pa 1976). 1999 Apr. 15; 24(8):755-62. doi: 10.1097/00007632-199904150-00005. PMID: 10222525
Wong, J., Sampson, S. L., Bell-Briones, H., Ouyang, A., Lazar, A. A., Lotz, J. C., Fields, A. J., 2019. Nutrient supply and nucleus pulposus cell function: effects of the transport properties of the cartilage endplate and potential implications for intradiscal biologic therapy. Osteoarthritis and Cartilage 27, 956-964. doi.org/10.1016/j.joca.2019.01.013

TABLE 1
Human cadaver demographic data and lumbar disc allocation to study groups

| | Age | Sex | BMI | Control buffer | | Collagenase | |
|---|---|---|---|---|---|---|---|
| Donor # | (yrs) | (F/M) | (Kg/m\hat{ }2) | level | Pfirrmann Grade | level | Pfirrmann Grade |
| Donor 1 | 56 | F | 18.3 | L2-L3 | III | L3-L4 | III |
| Donor 2 | 57 | F | 24.8 | L4-L5 | III | L3-L4 | III |
| Donor 3 | 25 | M | 23.7 | L3-L4 | II | L4-L5 | II |
| Donor 4 | 61 | M | 27.4 | L3-L4 | III | L4-L5 | III |
| Donor 5 | 63 | F | 21.1 | L4-L5 | III | L3-L4 | III |

TABLE 2
Bovine coccygeal (CC) disc allocation to study groups

| Steer # | Sham | Control buffer | Collagenase |
|---|---|---|---|
| Bovine 1 | CC3 | CC1 | CC2 |
| Bovine 2 | CC1 | CC2 | CC3 |
| Bovine 3 | CC2 | CC3 | CC1 |

TABLE 3
Effects of intradiscal treatment on sGAG content
(μg/mg dry) at the NP different positions

| Treatment | Position A | Position B | Position C | Position D |
|---|---|---|---|---|
| Control | 231.9 ± 107.4^{a} | 222.9 ± 133.2 | 225.3 ± 107.6 | 223.1 ± 95.4 |
| Collagenase | 207.9 ± 133.8 | 241.3± 132.9 | 218.8 ± 105.4 | 245.2 ± 98.2 |

### Example 2

CEP Treatment with Full-Length MMP-8 Reduces Collagen Content and Increases Small-Solute Transport

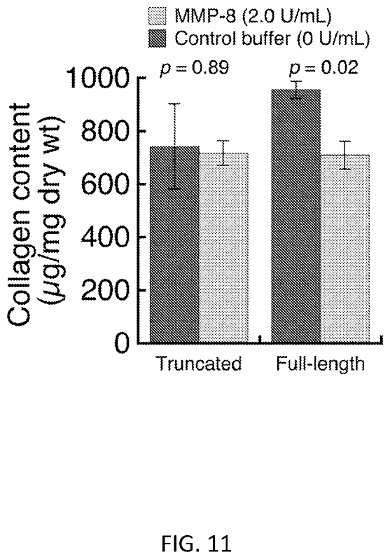

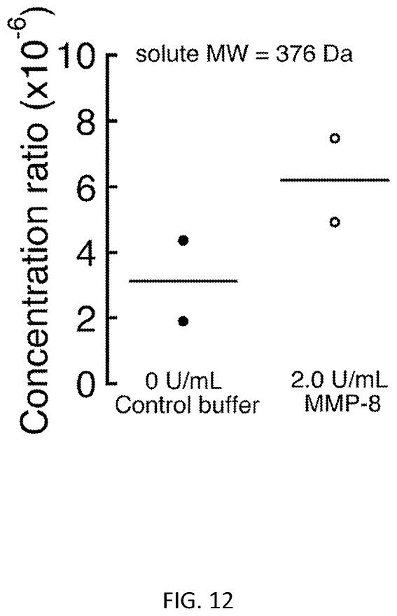

To test an enzyme with collagen-binding ability, we used full-length recombinant human MMP-8 (MMP8-1132H; Creative BioMart). Human CEPs from different donors were treated for 18 hr at 37 °C with equivalent doses of each form of the enzyme (2 U/mL in 0.1 mL of reaction buffer). Results showed that collagen content was 25.8% lower in the site-matched CEP tissues treated with full-length, but not truncated MMP-8 (FIG. 11). Conversely, sGAG was 14.8% lower in the CEPs treated with truncated MMP-8, but not full-length MMP-8 (p=0.89). These data imply that truncated MMP-8 mainly reduces aggrecan or sGAG in the CEP whereas full-length MMP-8 mainly reduces collagen. Treatment with full-length MMP-8 also improved small-solute transport. Briefly, CEPs were placed into a custom permeameter whose upstream inlet was connected to a pressurized solute reservoir (fluorescein, 376 Da). Fluid and solutes that passed through the CEP were collected downstream at 80 min (steady-state), assayed for solute concentration (C_{downstream}), and normalized to upstream values (C=C_{downstream}/Cᵤₚₛₜᵣₑₐₘ). This validated approach enables comparison between solutes and treatments [3]. In our pilot studies, fluorescein concentration ratios, C, from one patient were ~2-fold higher in site-matched biopsies treated with full-length MMP-8 (2.0 U/mL) than those treated with control buffer (FIG. 12).

### Example 3

CEP Treatment with Full-Length MMP-8 has Few Direct and Indirect Off-Target Matrix Effects

We used mass spectrometry to profile the peptides in untreated (n=4 patients) and MMP-8-treated (n=2 patients) human CEP biopsies, including the “releasates” from CEP proteolysis by MMP-8 (n=4 patients). We identified 8,664 peptides corresponding to 250 unique proteins in the untreated CEP samples and 5,653 peptides belonging to 201 unique proteins in the treated CEP samples. The exogenous MMP-8 was identified in every treated sample, but not in any of the untreated samples, suggesting good penetration of the enzyme into the tissue. Present among the proteins discovered were known targets of full-length MMP-8, including collagen II (COL2A1). In the releasates from CEPs treated with full-length MMP-8, we identified 288 peptides belonging to 24 unique proteins, the most abundant being collagen II (>3× more peptides than the next most abundant). Also identified in the releasates were two collagen subtypes (COL3A1, COL5A2) that were not identified in the treated or untreated CEPs. This collagen enrichment suggests productive collagenolytic activity of MMP-8. Finally, of the proteins identified in untreated CEPs, only 16 (6.4%) were found in the releasates following treatment, which supports the specificity of full-length MMP-8 activity with few direct and indirect off-target effects on the matrix. To our knowledge, this is the first proteomic analysis of human CEPs.

### Example 4

CEP Cells Exhibit Low Sensitivity to Matrix Fragments from MMP-8 Proteolysis

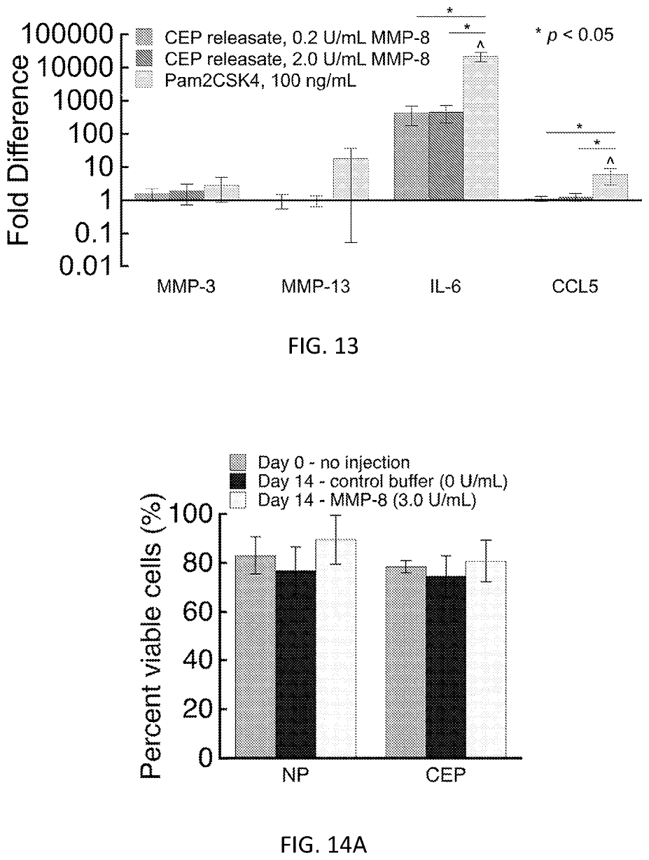

We tested CEP cells' response to the matrix fragments from proteolysis by MMP-8 using cell culture. Briefly, CEP cells were isolated from surgical waste tissues collected from five patients (3/2 female/male; 64.6±4.1 years-old), expanded to passage 3, and then cultured for 72 hr in four conditions: basal growth medium (negative control); medium supplemented with 100 ng/mL Pam2CSK4 (TLR2/6 agonist, positive control); and medium supplemented with matrix fragments (“releasates”) resulting from 12 hr treatment of patient-matched CEP tissues with 0.2 or 2.0 U/mL of full-length MMP-8. After culture, media was assayed by ELISA. We found mostly minimal effects of the releasates from MMP-8, whereas the TLR2/6 agonist (Pam2CSK4) significantly increased production of inflammatory cytokines (IL-6) and chemokines (CCL5) in the CEP cells (FIG. 13).

### Example 5

Intradiscal Treatment of the CEP with Full-Length MMP-8 Increases NP Cell Viability

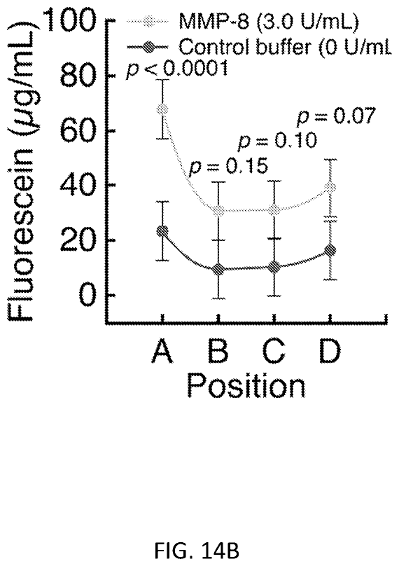

We also tested CEP treatment in a bovine organ culture model. The superior CEPs of discs from two fresh bovine tails (3 discs/tail) were treated intradiscally with full-length MMP-8 (3.0 U/mL) or control buffer (0 U/mL, HBSS) in alginate carrier (50 μL) as described above. Discs were then cultured in growth medium (low glucose DMEM with 5% FBS and NaCl) for two weeks at 21%/5% O₂/CO₂ and 37 °C with daily compressive loading (0.2 MPa; 8 hr/day) via porous platens. After culture, cell viability was measured with a confocal microscope after staining with a Live/Dead assay (1 disc/group), and fluorescein transport was measured after biomechanical testing as described above [2](2 discs/group). In the disc with the MMP-8-treated CEP, cell viability in the central NP was 13% higher (89% vs. 76%, FIG. 14A), consistent with up to 3-fold higher fluorescein concentrations near the treated CEP (FIG. 14B). Although requiring confirmation in additional discs, these results match our findings in the diffusion chambers [10] and suggest that the zone of improvement in nutrient transport induced by the CEP treatments is sufficient to improve cell viability in the central NP. More generally, these results have important translational implications because they suggest that one-time treatments to reduce impediments to solute transport through the CEP can affect long-term improvements in NP cell viability.

What is claimed is:

1. A method of treating intervertebral disc degeneration in a subject in need thereof, the method comprising administering a therapeutically effective amount of one or more matrix-modifying enzymes locally to a cartilage endplate (CEP).

2. The method of claim 1, wherein treatment increases permeability of the CEP and intervertebral disc nutrient transport.

3. The method of claim 1 or 2, wherein the one or more matrix-modifying enzymes have proteolytic activity against a collagen, a proteoglycan, or a combination thereof.

4. The method of claim 3, wherein the one or more matrix-modifying enzymes comprise a collagenase.

5. The method of claim 4, wherein the collagenase has proteolytic activity against collagen I, collagen II, collagen III, collagen IV, or any combination thereof.

6. The method of claim 5, wherein the collagenase is collagenase P, matrix metalloproteinase 8 (MMP8), or a combination thereof.

7. The method of claim 6, wherein the MMP8 is full-length MMP8.

8. The method of any one of claims 1-7, wherein the one or more matrix-modifying enzymes are encapsulated in a hydrogel.

9. The method of claim 8, wherein the hydrogel comprises alginate.

10. The method of claim 9, wherein the alginate concentration in the hydrogel ranges from 0.5 to 10 percentage by weight (wt %).

11. The method of claim 10, wherein the alginate concentration in the hydrogel is about 1 wt %.

12. The method of any one of claims 9-11, wherein the alginate is at least partially oxidized.

13. The method of claim 12, wherein about 4% to about 10% of the alginate is oxidized.

14. The method of any one of claims 8-13, wherein the hydrogel sustains delivery of the matrix-modifying enzyme for at least 2 days.

15. The method of any one of claims 1-7, wherein the one or more matrix-modifying enzymes are encapsulated in a liposome or conjugated to the surface of a liposome.

16. The method of claim 15, wherein the liposome comprises a phospholipid.

17. The method of claim 16, wherein the phospholipid is phosphatidylcholine or phosphatidylethanolamine.

18. The method of any one of claims 15-17, wherein the liposome further comprises cholesterol.

19. The method of any one of claims 15-18, wherein the liposome further comprises polyethylene glycol.

20. The method of any one of claims 1-19, wherein the one or more matrix-modifying enzymes are administered in a volume sufficiently small such that intradiscal pressure is not substantially increased.

21. The method of claim 20, wherein the one or more matrix-modifying enzymes are administered in a volume of no more than 50 μL.

22. The method of any one of claims 1-21, wherein the one or more matrix-modifying enzymes are administered into or adjacent to the CEP.

23. The method of any one of claims 1-22, wherein the one or more matrix-modifying enzymes are administered with an epidural needle.

24. The method of claim 23, wherein the epidural needle has a curved end such that the the matrix-modifying enzyme exits the epidural needle laterally at about a 45-degree angle.

25. The method of any one of claims 1-24, further comprising using medical imaging to guide said administering the one or more matrix-modifying enzymes locally to the CEP or for selection of which intervertebral discs may benefit from treatment with the one or more matrix-modifying enzymes.

26. The method of claim 25, wherein said administering the one or more matrix-modifying enzymes locally to the CEP is performed with fluoroscopic guidance, computed tomographic (CT) guidance, combined CT-fluoroscopic guidance, or ultrasound guidance.

27. The method of any one of claims 1-26, wherein the one or more matrix-modifying enzymes are administered locally to the CEP without exposing the nucleus pulposus to the one or more matrix-modifying enzymes.

28. The method of any one of claims 1-27, further comprising administering a growth factor.

29. The method of any one of claims 1-28, further comprising administering an analgesic agent or an anti-inflammatory agent.

30. The method of any one of claims 1-29, wherein multiple cycles of treatment are administered to the subject.

31. A composition comprising one or more matrix-modifying enzymes for use in a method of treating intervertebral disc degeneration.

32. The composition of claim 31, further comprising a pharmaceutically acceptable excipient.

33. The composition of claim 31 or 32, wherein the one or more matrix-modifying enzymes comprise a collagenase.

34. The composition of claim 33, wherein the collagenase has proteolytic activity against collagen I, collagen II, collagen III, collagen IV, or any combination thereof.

35. The composition of claim 34, wherein the collagenase is collagenase P, matrix metalloproteinase 8 (MMP8), or a combination thereof.

36. The composition of claim 35, wherein the MMP8 is full-length MMP8.

37. The composition of any one of claims 31-36, wherein the one or more matrix-modifying enzymes are encapsulated in a hydrogel.

38. The composition of claim 37, wherein the hydrogel comprises alginate.

39. The composition of claim 38, wherein the alginate concentration in the hydrogel ranges from 0.5 to 10 percentage by weight (wt %).

40. The composition of claim 39, wherein the alginate concentration in the hydrogel is about 1 wt %.

41. The composition of any one of claims 38-40, wherein the alginate is at least partially oxidized.

42. The composition of claim 41, wherein about 4% to about 10% of the alginate is oxidized.

43. The composition of any one of claims 37-42, wherein the hydrogel sustains delivery of the matrix-modifying enzyme for at least 2 days.

44. The composition of any one of claims 31-36, wherein the one or more matrix-modifying enzymes are encapsulated in a liposome or conjugated to the surface of a liposome.

45. The composition of claim 44, wherein the liposome comprises a phospholipid.

46. The composition of claim 45, wherein the phospholipid is phosphatidylcholine or phosphatidylethanolamine.

47. The composition of any one of claims 44-46, wherein the liposome further comprises cholesterol.

48. The composition of any one of claims 44-47, wherein the liposome further comprises polyethylene glycol.

49. The composition of any one of claims 31-48, further comprising a growth factor.

50. The composition of any one of claims 31-49, further comprising an analgesic agent or an anti-inflammatory agent.

51. A method of increasing intervertebral disc solute transport in a subject in need thereof, the method comprising administering an effective amount of one or more matrix-modifying enzymes locally to a cartilage endplate (CEP).

52. The method of claim 51, wherein said administering increases permeability of the CEP and intervertebral disc nutrient transport.

53. The method of claim 51 or 52, wherein the one or more matrix-modifying enzymes have proteolytic activity against a collagen, a proteoglycan, or a combination thereof.

54. The method of claim 53, wherein the one or more matrix-modifying enzymes comprise a collagenase.

55. The method of claim 54, wherein the collagenase has proteolytic activity against collagen I, collagen II, collagen III, collagen IV, or any combination thereof.

56. The method of claim 55, wherein the collagenase is collagenase P, matrix metalloproteinase 8 (MMP8), or a combination thereof.

57. The method of claim 56, wherein the MMP8 is full-length MMP8.

58. The method of any one of claims 51-57, wherein the one or more matrix-modifying enzymes are encapsulated in a hydrogel.

59. The method of claim 58, wherein the hydrogel comprises alginate.

60. The method of claim 59, wherein the alginate concentration in the hydrogel ranges from 0.5 to 10 percentage by weight (wt %).

61. The method of claim 60, wherein the alginate concentration in the hydrogel is about 1 wt %.

62. The method of any one of claims 59-61, wherein the alginate is at least partially oxidized.

63. The method of claim 62, wherein about 4% to about 10% of the alginate is oxidized.

64. The method of any one of claims 58-63, wherein the hydrogel sustains delivery of the matrix-modifying enzyme for at least 2 days.

65. The method of any one of claims 51-57, wherein the one or more matrix-modifying enzymes are encapsulated in a liposome or conjugated to the surface of a liposome.

66. The method of claim 65, wherein the liposome comprises a phospholipid.

67. The method of claim 66, wherein the phospholipid is phosphatidylcholine or phosphatidylethanolamine.

68. The method of any one of claims 65-67, wherein the liposome further comprises cholesterol.

69. The method of any one of claims 65-68, wherein the liposome further comprises polyethylene glycol.

70. The method of any one of claims 51-69 wherein the one or more matrix-modifying enzymes are administered in a volume sufficiently small such that intradiscal pressure is not substantially increased.

71. The method of claim 70, wherein the one or more matrix-modifying enzymes are administered in a volume of no more than 50 μL.

72. The method of any one of claims 51-71, wherein the one or more matrix-modifying enzymes are administered into or adjacent to the CEP.

73. The method of any one of claims 51-72, wherein the one or more matrix-modifying enzymes are administered with an epidural needle.

74. The method of claim 73, wherein the epidural needle has a curved end such that the the one or more matrix-modifying enzymes exit the epidural needle laterally at about a 45-degree angle.

75. The method of any one of claims 51-74, further comprising using medical imaging to guide said administering the one or more matrix-modifying enzymes locally to the CEP or selection of which intervertebral discs may benefit from treatment with the one or more matrix-modifying enzymes.

76. The method of claim 75, wherein said administering the one or more matrix-modifying enzymes locally to the CEP is performed with fluoroscopic guidance, computed tomographic (CT) guidance, combined CT-fluoroscopic guidance, or ultrasound guidance.

77. The method of any one of claims 51-76, wherein the one or more matrix-modifying enzymes are administered locally to the CEP without exposing the nucleus pulposus to the one or more matrix-modifying enzymes.

78. The method of any one of claims 51-77, further comprising administering a growth factor.

79. The method of any one of claims 51-78, further comprising administering an analgesic agent or an anti-inflammatory agent.

80. The method of any one of claims 51-79, wherein prior to said administering the one or more matrix-modifying enzymes locally to the CEP, the method further comprises detecting if intervertebral disc solute transport is impeded in the CEP to determine if the subject will benefit from the treatment.
